(19) United States
(12) Patent Application Publication
(10) Pub. No.: US 2026/0226136 A1
(43) Pub. Date: Aug. 6, 2026

(54) T CELL RECEPTORS TARGETING MUTATIONS IN RNA SPLICING FACTORS

(71) Applicant: University of Pittsburgh - Of the Commonwealth System of Higher Education, Pittsburgh, PA (US)

(72) Inventors: Udai Shankar Kammula, Pittsburgh, PA (US); Ghanshyam Singh Yadav, Pittsburgh, PA (US); Chetana Bhaskarla, Pittsburgh, PA (US)

(21) Appl. No.: 19/577,638

(22) Filed: Mar. 25, 2026

Related U.S. Application Data
(62) Division of application No. 17/779,711, filed on Nov. 25, 2020, now Pat. No. 12,612,444.
(60) Provisional application No. 62/940,725, filed on Nov. 26, 2019.

(51) Int. Cl. C07K 14/725; A61K 40/11; A61K 40/32; A61K 40/42; A61P 35/00; C12N 15/86

(52) U.S. Cl. CPC C07K 14/7051; A61K 40/11; A61K 40/32; A61K 40/42; A61P 35/00; C12N 15/86; C07K 2319/50; C12N 2740/15043

(57) ABSTRACT

This document provides isolated immune cells that include an exogenous T cell receptor (TCR) having affinity for a splicing factor 3B subunit 1A (SF3B1) peptide as well as methods and materials for making such immune cells. For example, isolated immune cells that included an exogenous TCR having affinity for a mutant SF3B1 peptide, methods and materials for making such immune cells, and methods and materials for using such immune cells to treat mammals (e.g., a human having cancer) are provided.

### CLAIM OF PRIORITY

This application is a Divisional of U.S. application Ser. No. 17/779,711, filed May 25, 2022, which is a National Stage application under 35 U.S.C. § 371 of International Application No. PCT/US2020/062164, having an International Filing Date of Nov. 25, 2020, which claims priority to U.S. Provisional Application No. 62/940,725, filed on Nov. 26, 2019, the entire contents of which each are hereby incorporated by reference.

### SEQUENCE LISTING

This application contains a sequence listing that has been submitted electronically as XML file named 45049-0034002_SL_ST26.xml. The XML file, created on Apr. 8, 2026, is 127,231 bytes in size. The material in the XML file is hereby incorporated by reference in its entirety.

### BACKGROUND

### 1. Technical Field

This document relates to isolated immune cells that include an exogenous T cell receptor (TCR) having affinity for a splicing factor 3B subunit 1A (SF3B1) peptide as well as methods and materials for making such immune cells. For example, this document provides isolated immune cells that included an exogenous TCR having affinity for a mutant SF3B1 peptide, methods and materials for making such immune cells, and methods and materials for using such immune cells to treat mammals (e.g., a human having cancer).

### 2. Background Information

RNA splicing is a fundamental process in eukaryotes, which is carried out by the splicing machinery (spliceosome). Splicing factor 3b subunit 1 (SF3B1) is a component of the U2 small nuclear ribonucleoproteins complex, which is involved in RNA alternative splicing. Mutant SF3B1 results in abnormal splicing of several genes, primarily due to misrecognition of 3′ splice sites. Many of the resulting aberrant mRNAs undergo nonsense-mediated mRNA decay (NMD), leading to reduced gene expression. See, for example, Giuseppe Liberante, et al., Sci. Rep., 9:2678 (2019). Missense mutations in SF3B1 are associated with the development and progression of multiple malignant diseases, including myelodysplastic syndromes, breast cancer, and chronic lymphocytic leukemia (CLL).

### SUMMARY

This document is based, at least in part, on the discoveries of T cell receptors (TCRs) having affinity for a SF3B1 (Splicing Factor 3B Subunit 1A) peptide, e.g., a mutant SF3B1 peptide. As described herein, TCRs provided herein bind to a mutant SF3B1 peptide (within the context of an MHC molecule such as DRB3) have an alpha chain and a beta chain. In some embodiments, the alpha chain includes (i) the amino acid sequences set forth in SEQ ID NO:10, SEQ ID NO:11, and SEQ ID NO:12, or (ii) the amino acid sequences set forth in SEQ ID NO:10 and SEQ ID NO:11 with no more than one amino acid modification, and the amino acid sequence set forth in SEQ ID NO:12 with no more than two amino acid modifications, and the beta chain includes (i) the amino acid sequences set forth in SEQ ID NO: 1, SEQ ID NO:2, and SEQ ID NO:3, or (ii) the amino acid sequences set forth in SEQ ID NO:1 and SEQ ID NO:2 with no more than one amino acid modification, and the amino acid sequence set forth in SEQ ID NO:3 with no more than two amino acid modifications. The amino acid modifications can be an amino acid substitution, an amino acid deletion, or an amino acid addition. In some embodiments, the alpha chain includes (i) the amino acid sequences set forth in SEQ ID NO:32, SEQ ID NO:33, and SEQ ID NO:34, or (ii) the amino acid sequences set forth in SEQ ID NO:32 and SEQ ID NO:33 with no more than one amino acid modification and the amino acid sequence set forth in SEQ ID NO:34 with no more than two amino acid modifications, and the beta chain includes (i) the amino acid sequences set forth in SEQ ID NO:23, SEQ ID NO:24, and SEQ ID NO:25, or (ii) the amino acid sequences set forth in SEQ ID NO:23 and SEQ ID NO:24 with no more than one amino acid modification, and the amino acid sequence set forth in SEQ ID NO:25 with no more than two amino acid modifications. The amino acid modifications can be an amino acid substitution, an amino acid deletion, or an amino acid addition.

Immune cells (e.g., T cells) modified to include an exogenous TCR having affinity for a SF3B1 peptide (e.g., a mutant SF3B1 peptide) as described herein can be used to treat a mammal having cancer. Mutations in SF3B1 are commonly found, for example, in uveal melanoma, chronic lymphocytic leukemia, myelodysplastic syndromes, and breast cancer.

In one aspect, this document features an isolated immune cell (e.g., a T cell) comprising an exogenous TCR having affinity for a splicing factor 3B subunit 1A (SF3B1) peptide. The exogenous TCR includes an alpha chain and a beta chain, where the alpha chain includes (i) the amino acid sequences set forth in SEQ ID NO:10, SEQ ID NO:11, and SEQ ID NO:12, or (ii) the amino acid sequences set forth in SEQ ID NO:10 and SEQ ID NO: 11 with no more than one amino acid modification and the amino acid sequence set forth in SEQ ID NO:12 with no more than two amino acid modifications, wherein the amino acid modifications are selected from the group consisting of amino acid substitutions, amino acid deletions, and amino acid additions, and the beta chain includes (i) the amino acid sequences set forth in SEQ ID NO: 1, SEQ ID NO:2, and SEQ ID NO:3, or (ii) the amino acid sequences set forth in SEQ ID NO:1 and SEQ ID NO:2 with no more than one amino acid modification and the amino acid sequence set forth in SEQ ID NO:3 with no more than two amino acid modifications, wherein the amino acid modifications are selected from the group consisting of amino acid substitutions, amino acid deletions, and amino acid additions.

In some cases, the alpha chain comprises an amino sequence having at least 80 percent identity, at least 90 percent identity, or at least 95 percent identity to the amino acid sequence set forth in SEQ ID NO:18 or SEQ ID NO:22. In some cases, the alpha chain includes the amino acid sequence set forth in SEQ ID NO: 10. In some cases, the alpha chain includes the amino acid sequence set forth in SEQ ID NO: 11. In some cases, the alpha chain includes the amino acid sequence set forth in SEQ ID NO: 12. In some cases, the alpha chain includes the amino acid sequence set forth in SEQ ID NO: 10, SEQ ID NO: 11, and SEQ ID NO:12.

In some cases, the beta chain comprises an amino acid sequence having at least 80 percent identity, at least 90 percent identity, or at least 95 percent identity to the amino acid sequence set forth in SEQ ID NO:9 or SEQ ID NO:20. In some cases, the beta chain includes the amino acid sequence set forth in SEQ ID NO: 1. In some cases, the beta chain includes the amino acid sequence set forth in SEQ ID NO:2. In some cases, the beta chain includes the amino acid sequence set forth in SEQ ID NO:3. In some cases, the beta chain comprises the amino acid sequence set forth in SEQ ID NO:1, SEQ ID NO: 2 and SEQ ID NO:3.

This document also features an isolated immune cell (e.g., a T cell) that includes an exogenous TCR having affinity for a SF3B1 peptide (e.g., a SF3B1 peptide comprising a histidine at the position corresponding to 625 in the human SF3B1 protein). The exogenous TCR includes an alpha chain and a beta chain, where the alpha chain includes (i) the amino acid sequences set forth in SEQ ID NO: 32, SEQ ID NO:33, and SEQ ID NO:34, or (ii) the amino acid sequences set forth in SEQ ID NO:32 and SEQ ID NO:33 with no more than one amino acid modification, and the amino acid sequence set forth in SEQ ID NO:34 with no more than two amino acid modifications, wherein the amino acid modifications are selected from the group consisting of amino acid substitutions, amino acid deletions, and amino acid additions; and the beta chain includes (i) the amino acid sequences set forth in SEQ ID NO:23, SEQ ID NO:24, and SEQ ID NO:25, or (ii) the amino acid sequences set forth in SEQ ID NO:23 and SEQ ID NO:24 with no more than one amino acid modification and the amino acid sequence set forth in SEQ ID NO:25 with no more than two amino acid modifications, wherein the amino acid modifications are selected from the group consisting of amino acid substitutions, amino acid deletions, and amino acid additions.

In some cases, the alpha chain includes an amino sequence having at least 80 percent identity, at least 90 percent identity, or at least 95 percent identity to the amino acid sequence set forth in SEQ ID NO:40 or SEQ ID NO:44. In some cases, the alpha chain includes the amino acid sequence set forth in SEQ ID NO:32. In some cases, the alpha chain includes the amino acid sequence set forth in SEQ ID NO:33. In some cases, the alpha chain includes the amino acid sequence set forth in SEQ ID NO:34. In some cases, the alpha chain includes the amino acid sequence set forth in SEQ ID NO:32, SEQ ID NO:33, and SEQ ID NO:34.

In some cases, the beta chain includes an amino acid sequence having at least 80 percent identity, at least 90 percent identity, or at least 95 percent identity to the amino acid sequence set forth in SEQ ID NO:31 or SEQ ID NO:42. In some cases, the beta chain includes the amino acid sequence set forth in SEQ ID NO:23. In some cases, the beta chain includes the amino acid sequence set forth in SEQ ID NO:24. In some cases, the beta chain includes the amino acid sequence set forth in SEQ ID NO:25. In some cases, the beta chain includes the amino acid sequence set forth in SEQ ID NO:23, SEQ ID NO: 24 and SEQ ID NO:25.

In any of the embodiments, the immune cell can be a T cell. Expression of the endogenous TCR alpha and beta chain coding sequences can be downregulated in the T cell.

This document also features a nucleic acid molecule that includes a nucleic acid sequence encoding an alpha chain of a TCR having affinity for a SF3B1 peptide and a nucleic acid sequence encoding a beta chain of the TCR. In some cases, the alpha chain includes (i) the amino acid sequences set forth in SEQ ID NO: 10, SEQ ID NO:11, and SEQ ID NO:12, or (ii) the amino acid sequences set forth in SEQ ID NO:10 and SEQ ID NO:11 with no more than one amino acid modification and the amino acid sequence set forth in SEQ ID NO:12 with no more than two amino acid modifications, wherein the amino acid modifications are selected from the group consisting of amino acid substitutions, amino acid deletions, and amino acid additions, and the beta chain includes (i) the amino acid sequences set forth in SEQ ID NO:1, SEQ ID NO:2, and SEQ ID NO:3, or (ii) the amino acid sequences set forth in SEQ ID NO:1 and SEQ ID NO:2 with no more than one amino acid modification and the amino acid sequence set forth in SEQ ID NO:3 with no more than two amino acid modifications, wherein the amino acid modifications are selected from the group consisting of amino acid substitutions, amino acid deletions, and amino acid additions. In some cases, the alpha chain includes (i) the amino acid sequences set forth in SEQ ID NO:32, SEQ ID NO:33, and SEQ ID NO: 34, or (ii) the amino acid sequences set forth in SEQ ID NO:32 and SEQ ID NO:33 with no more than one amino acid modification and the amino acid sequence set forth in SEQ ID NO: 34 with no more than two amino acid modifications, wherein the amino acid modifications are selected from the group consisting of amino acid substitutions, amino acid deletions, and amino acid additions; and the beta chain includes (i) the amino acid sequences set forth in SEQ ID NO:23, SEQ ID NO:24, and SEQ ID NO:25, or (ii) the amino acid sequences set forth in SEQ ID NO:23 and SEQ ID NO:24 with no more than one amino acid modification and the amino acid sequence set forth in SEQ ID NO:25 with no more than two amino acid modifications, wherein the amino acid modifications are selected from the group consisting of amino acid substitutions, amino acid deletions, and amino acid additions.

In some cases, the alpha chain comprises an amino sequence having at least 80 percent identity, at least 90 percent identity, or at least 95 percent identity to the amino acid sequence set forth in SEQ ID NO:18 or SEQ ID NO:22. In some cases, the alpha chain includes the amino acid sequence set forth in SEQ ID NO: 10. In some cases, the alpha chain includes the amino acid sequence set forth in SEQ ID NO: 11. In some cases, the alpha chain includes the amino acid sequence set forth in SEQ ID NO: 12. In some cases, the alpha chain includes the amino acid sequence set forth in SEQ ID NO: 10, SEQ ID NO: 11, and SEQ ID NO:12. In some cases, the beta chain comprises an amino acid sequence having at least 80 percent identity, at least 90 percent identity, or at least 95 percent identity to the amino acid sequence set forth in SEQ ID NO:9 or SEQ ID NO:20. In some cases, the beta chain includes the amino acid sequence set forth in SEQ ID NO: 1. In some cases, the beta chain includes the amino acid sequence set forth in SEQ ID NO:2. In some cases, the beta chain includes the amino acid sequence set forth in SEQ ID NO:3. In some cases, the beta chain comprises the amino acid sequence set forth in SEQ ID NO: 1, SEQ ID NO: 2 and SEQ ID NO:3.

In some cases, the alpha chain includes an amino sequence having at least 80 percent identity, at least 90 percent identity, or at least 95 percent identity to the amino acid sequence set forth in SEQ ID NO:40 or SEQ ID NO:44. In some cases, the alpha chain includes the amino acid sequence set forth in SEQ ID NO:32. In some cases, the alpha chain includes the amino acid sequence set forth in SEQ ID NO:33. In some cases, the alpha chain includes the amino acid sequence set forth in SEQ ID NO:34. In some cases, the alpha chain includes the amino acid sequence set forth in SEQ ID NO:32, SEQ ID NO:33, and SEQ ID NO:34. In some cases, the beta chain includes an amino acid sequence having at least 80 percent identity, at least 90 percent identity, or at least 95 percent identity to the amino acid sequence set forth in SEQ ID NO:31 or SEQ ID NO:42. In some cases, the beta chain includes the amino acid sequence set forth in SEQ ID NO:23. In some cases, the beta chain includes the amino acid sequence set forth in SEQ ID NO:24. In some cases, the beta chain includes the amino acid sequence set forth in SEQ ID NO:25. In some cases, the beta chain includes the amino acid sequence set forth in SEQ ID NO:23, SEQ ID NO:24 and SEQ ID NO:25.

In any of the embodiments, the nucleic acid can include a promotor 5′ of the nucleic acid sequence encoding the alpha chain and/or can include a promoter 5′ of the nucleic acid sequence encoding the beta chain. The promoter can be a viral 5′ long terminal repeat or a viral 3′ long terminal repeat.

In any of the embodiments, the nucleic acid can be a vector, e.g., a viral vector such as a retroviral vector or a lentiviral vector.

In any of the embodiments, the nucleic acid further can include a nucleic acid sequence encoding a linker. In some cases, the linker can be 3′ of the nucleic acid sequence encoding the alpha chain and 5′ of the nucleic acid sequence encoding the beta chain. In some cases, the linker can be 3′ of the nucleic acid sequence encoding the beta chain and 5′ of the nucleic acid sequence encoding the alpha chain. In some cases, the linker is a self-cleaving peptide (e.g., a self-cleaving peptide of a foot-and-mouth disease virus (FMDV), an equine rhinitis A virus (ERAVO, a Thosea asigna virus (TaV), or a porcine tescho virus-1 (PTV-1)). In some cases, the self-cleaving peptide is a P2A peptide. In some cases, the linker includes a furin cleavage site.

This document also features a method of making an immune cell (e.g., a T cell) that includes an exogenous TCR having affinity for a SF3B1 peptide. The method includes introducing, into the immune cell, a nucleic acid molecule that includes a nucleic acid sequence encoding an alpha chain of a TCR having affinity for a SF3B1 peptide and a nucleic acid sequence encoding a beta chain of the TCR, wherein the exogenous TCR is expressed from the nucleic acid in the immune cell.

In another aspect, this document features an isolated immune cell that includes a nucleic acid molecule and includes an exogenous TCR having affinity for a SF3B1 peptide. The nucleic acid molecule includes a nucleic acid sequence encoding an alpha chain of a TCR having affinity for a SF3BT peptide and a nucleic acid sequence encoding a beta chain of the TCR.

This document also features a population of cells that include at least one isolated immune cell, where the isolated immune cell includes a nucleic acid molecule and includes an exogenous TCR having affinity for a SF3B1 peptide. This document also features a pharmaceutical composition that includes the population of cells and a pharmaceutically acceptable carrier. A method for treating a mammal (e.g., a human) in need thereof also is provided that includes administering to the mammal an effective amount of the pharmaceutical composition. The mammal can have cancer (e.g., a cancer selected from the group consisting of melanoma, chronic lymphocytic leukemia, a myelodysplastic syndrome, and breast cancer). The immune cells can be autologous to the mammal.

In another aspect, this document features a method for providing a mammal (e.g., a human) with immune cells that include an exogenous TCR having affinity for a SF3BT peptide. The method includes administering, to the mammal, the population of cells. The mammal can have cancer (e.g., a cancer selected from the group consisting of melanoma, chronic lymphocytic leukemia, a myelodysplastic syndrome, and breast cancer). The immune cells can be autologous to the mammal.

A method for providing a mammal (e.g., a human) with cells comprising a TCR having affinity for a SF3B1 peptide also is provided. The method includes delivering, to the mammal, a nucleic acid molecule that includes a nucleic acid sequence encoding an alpha chain of a TCR having affinity for a SF3B1 peptide and a nucleic acid sequence encoding a beta chain of the TCR, wherein the nucleic acid is expressed in cells of the mammal. The mammal can have cancer (e.g., a cancer selected from the group consisting of melanoma, chronic lymphocytic leukemia, a myelodysplastic syndrome, and breast cancer). The immune cells can be autologous to the mammal.

In another aspect, this document features a method for treating a mammal (e.g., a human) having cancer. The method includes administering, to the mammal, a population of cells that include at least one isolated immune cell, where the isolated immune cell includes a nucleic acid molecule and includes an exogenous TCR having affinity for a SF3B1 peptide or administering a nucleic acid molecule that includes a nucleic acid sequence encoding an alpha chain of a TCR having affinity for a SF3B1 peptide and a nucleic acid sequence encoding a beta chain of the TCR. The immune cells can be autologous to the mammal.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A contains an alignment of the mutant SF3B1 peptide (SEQ ID NO:61) containing a histidine at the position corresponding to 625 in the human SF3B1 protein and a wild-type SF3B1 peptide (SEQ ID NO:79) containing an arginine at the position corresponding to 625 in the human SF3B1 protein.

FIG. 1B contains the nucleotide sequence encoding the TRBV2-01+TRAV12-02 chimeric TCR (SEQ ID NO:49) and the amino acid sequence of the TRBV2-01+TRAV12-02 chimeric TCR (SEQ ID NO:50).

FIG. 2 contains an exemplary codon optimized nucleotide sequence encoding the TRBV2-01+TRAV12-02 chimeric TCR (SEQ ID NO:51) and the amino acid sequence of the TRBV2-01+TRAV12-02 chimeric TCR (SEQ ID NO:50).

FIG. 3 contains the nucleotide sequence encoding the human TRBV2-01+TRAV12-02 TCR (SEQ ID NO:52) and the amino acid sequence of the human TRBV2-01+TRAV12-02 TCR (SEQ ID NO:53).

FIG. 4 contains an exemplary codon optimized nucleotide sequence encoding a human TRBV2-01+TRAV12-02 TCR (SEQ ID NO:54) and the amino acid sequence of the human TRBV2-01+TRAV12-02 TCR (SEQ ID NO:53).

FIG. 5 contains the nucleotide sequence encoding a TRBV27-01+TRAV13-01 chimeric TCR (SEQ ID NO:55) and the amino acid sequence of the TRBV27-01+TRAV13-01 chimeric TCR (SEQ ID NO:56).

FIG. 6 contains an exemplary codon optimized nucleotide sequence encoding the TRBV27-01+TRAV13-01 chimeric TCR (SEQ ID NO:57) and the amino acid sequence of the TRBV27-01+TRAV13-01 chimeric TCR (SEQ ID NO:56).

FIG. 7 contains the nucleotide sequence encoding a human TRBV27-01+TRAV13-01 TCR (SEQ ID NO:58) and the amino acid sequence of the human TRBV27-01+TRAV13-01 TCR (SEQ ID NO:59).

FIG. 8 contains an exemplary codon optimized nucleotide sequence encoding the human TRBV27-01+TRAV13-01 (SEQ ID NO:60) TCR and the amino acid sequence of the human TRBV27-01+TRAV13-01 TCR (SEQ ID NO:59).

FIG. 9 contains a graph of interferon gamma (IFNγ) (pg/mL) in the supernatant after TCR-transduced T cells were co-cultured overnight with dendritic cells expressing wildtype (wt) SF3B1 (SEQ ID NO:79) or mutated forms of SF3B1 (R625H (SEQ ID NO:61), R625G (SEQ ID NO:89), R625L (SEQ ID NO:90), or R625C (SEQ ID NO:91)).

FIG. 10 contains the amino acid sequences set forth in Table 2.

FIG. 11A contains the nucleotide sequence encoding the beta chain of TRBV2-01+TRAV12-02 chimeric TCR (SEQ ID NO:92), with human beta variable region in larger font, CDR1, CDR2, CDR3 underlined, and mouse beta constant in italics.

FIG. 11B contains the nucleotide sequence encoding the alpha chain of TRBV2-01+TRAV12-02 chimeric TCR (SEQ ID NO:93), with human alpha variable region in larger font, CDR1, CDR2, CDR3 underlined, and mouse alpha constant in italics.

FIG. 12A contains the codon optimized nucleotide sequence encoding the beta chain of TRBV2-01+TRAV12-02 chimeric TCR (SEQ ID NO:94), with human beta variable region in larger font, CDR1, CDR2, CDR3 underlined, and mouse beta constant in italics.

FIG. 12B contains the codon optimized nucleotide sequence encoding the alpha chain of TRBV2-01+TRAV12-02 chimeric TCR (SEQ ID NO:95), with human alpha variable region in larger font, CDR1, CDR2, CDR3 underlined, and mouse alpha constant in italics.

FIG. 13A contains the nucleotide sequence encoding the beta chain of TRBV2-01+TRAV12-02 human TCR (SEQ ID NO:96), with human beta variable region in larger font, CDR1, CDR2, CDR3 underlined, and human beta constant in italics.

FIG. 13B contains the nucleotide sequence encoding the alpha chain of TRBV2-01+TRAV12-02 human TCR (SEQ ID NO:97), with human alpha variable region in larger font, CDR1, CDR2, CDR3 underlined, and human alpha constant in italics.

FIG. 14A contains the codon optimized nucleotide sequence encoding the beta chain of TRBV2-01+TRAV12-02 human TCR (SEQ ID NO:98), with human beta variable region in larger font, CDR1, CDR2, CDR3 underlined, and human beta constant in italics.

FIG. 14B contains the codon optimized nucleotide sequence encoding the alpha chain of TRBV2-01+TRAV12-02 human TCR (SEQ ID NO:99), with human alpha variable region in larger font, CDR1, CDR2, CDR3 underlined, and human alpha constant in italics.

FIG. 15A contains the nucleotide sequence encoding the beta chain of TRBV27-01+TRAV13-01 Chimeric TCR (SEQ ID NO: 100), with human beta variable region in larger font, CDR1, CDR2, CDR3 underlined, and mouse beta constant (codon optimized) in italics.

FIG. 15B contains the nucleotide sequence encoding the alpha chain of TRBV27-01+TRAV13-01 Chimeric TCR (SEQ ID NO: 101), with the human variable region in larger font, CDR1, CDR2, CDR3 underlined, and mouse alpha constant (codon optimized) in italics.

FIG. 16A contains the codon optimized nucleotide sequence encoding the beta chain of TRBV27-01+TRAV13-01 Chimeric TCR (SEQ ID NO:102), with human variable region in larger font, CDR1, CDR2, CDR3 underlined, and mouse beta constant in italics.

FIG. 16B contains the codon optimized nucleotide sequence encoding the alpha chain of TRBV27-01+TRAV13-01 Chimeric TCR (SEQ ID NO:103), with human variable region in larger font, CDR1, CDR2, CDR3 underlined, and mouse alpha constant in italics.

FIG. 17A contains the nucleotide sequence encoding the beta chain of TRBV27-01+TRAV13-01 human TCR (SEQ ID NO: 104), with human variable region in larger font, CDR1, CDR2, CDR3 underlined, and human beta constant in italics.

FIG. 17B contains the nucleotide sequence encoding the alpha chain of TRBV27-01+TRAV13-01 human TCR (SEQ ID NO: 105), with human variable region in larger font, CDR1, CDR2, CDR3 underlined, and human alpha constant in italics.

FIG. 18A contains the codon-optimized nucleotide sequence encoding the beta chain of TRBV27-01+TRAV13-01 human TCR (SEQ ID NO:106), with human variable region in larger font, CDR1, CDR2, CDR3 underlined, and human beta constant in italics.

FIG. 18B contains the codon-optimized nucleotide sequence encoding the alpha chain of TRBV27-01+TRAV13-01 human TCR (SEQ ID NO:107), with the human variable region in larger font, CDR1, CDR2, CDR3 underlined, and human alpha constant in italics.

### DETAILED DESCRIPTION

This document provides TCRs having affinity for a SF3B1 peptide, e.g., a mutant SF3B1 peptide (within the context of an MHC molecule presenting that SF3B1 peptide). The amino acid sequence of human SF3B1 is set forth in UniProtKB Accession No. 075533-1. See also Gene ID 23451 for the human SF3B1 gene. Mutations in SF3B1 are commonly found, for example, in uveal melanoma, chronic lymphocytic leukemia, myelodysplastic syndromes, and breast cancer. In some cases, the TCRs provided herein can have specific affinity for a mutant SF3B1 peptide (e.g., a mutant SF3B1 peptide comprising a histidine at the position corresponding to 625 in the human SF3B1 protein) within the context of an MHC molecule (e.g., DRB3) presenting that SF3B1 peptide. For example, the TCRs provided herein, and cells expressing such TCRs (e.g., immune cells), can have reactivity against a mutant SF3B1 peptide comprising a histidine at the position corresponding to 625 in the human SF3B1 protein without having reactivity to the wild-type of SF3B1 (e.g., having an arginine at the position corresponding to 625 in the human SF3B1 protein) or other mutants at the position corresponding to 625 in the human SF3B1 protein (e.g., having a glycine, cysteine, or leucine at the position corresponding to 625 in the human SF3B1 protein). See FIG. TA for an alignment of a mutant SF3B1 peptide (SEQ ID NO:61) comprising a histidine at the position corresponding to 625 in the human SF3B1 protein and a wild-type SF3B1 peptide (SEQ ID NO:79) having an arginine at the position corresponding to 625 in the human SF3B1 protein.

A mutant SF3B1 peptide can be from 8 to 30 amino acids in length. Examples of mutant SF3B1 peptides that can be recognized by a TCR provided herein are set forth in Table 1.

TABLE 1
Exemplary mutant SF3B1 peptides

| Sequence | SEQ ID NO: |
|---|---|
| MRPDIDNMDEYVHNTTARAFAVVAS | 61 |
| RPDIDNMDEYVHNTTARAFAVVAS | 62 |
| PDIDNMDEYVHNTTARAFAVVAS | 63 |
| DIDNMDEYVHNTTARAFAVVAS | 64 |
| IDNMDEYVHNTTARAFAVVAS | 65 |
| DNMDEYVHNTTARAFAVVAS | 66 |
| NMDEYVHNTTARAFAVVAS | 67 |
| MDEYVHNTTARAFAVVAS | 68 |
| DEYVHNTTARAFAVVAS | 69 |
| EYVHNTTARAFAVVAS | 70 |
| MRPDIDNMDEYVHNTTA | 71 |
| MRPDIDNMDEYVHNTTAR | 72 |
| MRPDIDNMDEYVHNTTARA | 73 |
| MRPDIDNMDEYVHNTTARAF | 74 |
| MRPDIDNMDEYVHNTTARAFA | 75 |
| MRPDIDNMDEYVHNTTARAFAV | 76 |
| MRPDIDNMDEYVHNTTARAFAVV | 77 |
| MRPDIDNMDEYVHNTTARAFAVVA | 78 |

For example, immune cells that include an exogenous TCR provided herein can be used to treat mammals (e.g., humans having cancer) who harbor the same or similar SF3B1 mutations. For example, immune cells that include an exogenous TCR provided herein can be used to treat melanoma (e.g., uveal melanoma) and/or other cancers such as chronic lymphocytic leukemia, a myelodysplastic syndrome, or breast cancer.

A TCR is a heterodimeric cell surface protein and is involved in mediating signal transduction. The extracellular portion of a native heterodimeric TCR includes two polypeptide chains, each of which has a membrane-proximal constant domain, and a membrane-distal variable domain. The variable domains contain highly polymorphic loops analogous to the complementarity determining regions (CDRs) of antibodies.

In general, the TCRs provided herein include an alpha chain polypeptide and a beta chain polypeptide, where the alpha chain and the beta chain each include three CDRs and a constant region. In some cases, the alpha chain polypeptide can include a CDR1 having the amino acid sequence set forth in SEQ ID NO:10 (or a variant with one, two, or three amino acid modifications), a CDR2 having the amino acid sequence set forth in SEQ ID NO:11 (or a variant with one, two, or three amino acid modifications), and a CDR3 having the amino acid sequence set forth in SEQ ID NO: 12 (or a variant with one, two, or three amino acid modifications), and the beta chain polypeptide can include a CDR1 having the amino acid sequence set forth in SEQ ID NO:1 (or a variant with one, two, or three amino acid modifications), a CDR2 having the amino acid sequence set forth in SEQ ID NO:2 (or a variant with one, two, or three amino acid modifications), and a CDR3 having the amino acid sequence set forth in SEQ ID NO:3 (or a variant with one, two, or three amino acid modifications). In some cases, the alpha chain polypeptide can include a CDR1 having the amino acid sequence set forth in SEQ ID NO:32 (or a variant with one, two, or three amino acid modifications), a CDR2 having the amino acid sequence set forth in SEQ ID NO:33 (or a variant with one, two, or three amino acid modifications), and a CDR3 having the amino acid sequence set forth in SEQ ID NO:34 (or a variant with one, two, or three amino acid modifications), and the beta chain polypeptide can include a CDR1 having the amino acid sequence set forth in SEQ ID NO:23 (or a variant with one, two, or three amino acid modifications), a CDR2 having the amino acid sequence set forth in SEQ ID NO:24 (or a variant with one, two, or three amino acid modifications), and a CDR3 having the amino acid sequence set forth in SEQ ID NO:25 (or a variant with one, two, or three amino acid modifications).

Examples of amino acid modifications with respect to the amino acid sequence set forth in SEQ ID NOs:1, 2, 3, 10, 11, 12, 23, 24, 25, 32, 33, and 34 include, without limitation, amino acid substitutions, amino acid deletions, and amino acid additions. In some cases, a variant of SEQ ID NOs:3, 12, 25, or 34 can include no more than one amino acid modification. Amino acid modifications can be made, for example, to improve the binding and/or contact with the SF3B1 peptide.

In some cases, an amino acid substitution that can be engineered into a TCR containing the sequence set forth in SEQ ID NO:1, 2, 3, 10, 11, 12, 23, 24, 25, 32, 33, or 34 can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution that can be engineered into a TCR containing the sequence set forth in SEQ ID NO:1, 2, 3, 10, 11, 12, 23, 24, 25, 32, 33, or 34 can be a non-conservative amino acid substitution. Non-conservative amino acid substitutions can be made by substituting one amino acid residue for another amino acid residue having a dis-similar side chain. Examples of non-conservative substitutions that can be used as described herein include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating amino acid sequence variants can include site-specific mutagenesis or random mutagenesis (e.g., by PCR) of the nucleic acid encoding the alpha and/or beta chain polypeptide. See, for example, Zoller, Curr. Opin. Biotechnol. 3: 348-354 (1992). Both naturally occurring and non-naturally occurring amino acids (e.g., artificially-derivatized amino acids) can be used to generate amino acid sequence variants of the alpha and/or beta chain polypeptides provided herein.

In some cases, the constant region of an alpha chain polypeptide of a TCR provided herein can have an amino acid sequence having at least 80% identity to the amino acid sequence set forth in SEQ ID NO:17 or SEQ ID NO:21 For example, the constant region of an alpha chain polypeptide of a TCR provided herein can have an amino acid sequence having at least 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:17 or SEQ ID NO:21.

In some cases, the variable region of an alpha chain polypeptide of a TCR provided herein can have an amino acid sequence having at least 80% identity to the amino acid sequence set forth in SEQ ID NO:45 provided that the alpha chain includes the appropriate combination of CDRs (or variants of the CDRs) described herein. For example, the variable region of an alpha chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:45, provided that it includes (a) SEQ ID NO: 10 (or SEQ ID NO:10 with no more than three, two, or one amino acid modifications), (b) SEQ ID NO:11 (or SEQ ID NO:11 with no more than three, two, or one amino acid modifications), and (c) SEQ ID NO: 12 (or SEQ ID NO: 12 with no more than three, two, or one amino acid modifications).

In some cases, the variable region of an alpha chain polypeptide of a TCR provided herein can include the amino acid sequence set forth in SEQ ID NO:45 (or a variant of SEQ ID NO:45 with one, two, three, four, five, six, seven, eight, nine, or 10 amino acid modifications, provided that the alpha chain includes the appropriate combination of CDRs (or variants of the CDRs) described herein). Amino acid modifications can be amino acid substitutions, amino acid deletions, or amino acid additions. Amino acid substitutions for SEQ ID NO:45 can be conservative amino acid substitutions or non-conservative amino acid substitutions as discussed above for SEQ ID NOs:1, 2, 3, 10, 11, 12, 23, 24, 25, 32, 33, and 34. In some cases, a variant of SEQ ID NO:45 can include no more than one, two, three, four, or five amino acid modifications.

In some cases, an alpha chain polypeptide of a TCR provided herein can have an amino acid sequence having at least 80% identity to the amino acid sequence set forth in SEQ ID NO:18 or SEQ ID NO:22, provided that the alpha chain includes the appropriate combination of CDRs (or variants of the CDRs) described herein. For example, an alpha chain of a TCR provided herein can have an amino acid sequence having at least 80%, 82%, 83%, 84%, 85%, 86%, 87%, 88%, 89%, 90%9, 91%, 92%, 93%, 94%, 95%, 96%, 97%, 98%, 99%, or 100% identity to the amino acid sequence set forth in SEQ ID NO: 18, provided that it includes (a) SEQ ID NO:10 (or SEQ ID NO:10 with no more than three, two, or one amino acid modifications), (b) SEQ ID NO:11 (or SEQ ID NO:11 with no more than three, two, or one amino acid modifications), and (c) SEQ ID NO: 12 (or SEQ ID NO: 12 with no more than three, two, or one amino acid modifications). In another example, an alpha chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:22, provided that it includes (a) SEQ ID NO:10 (or SEQ ID NO:10 with no more than three, two, or one amino acid modifications), (b) SEQ ID NO: 11 (or SEQ ID NO:11 with no more than three, two, or one amino acid modifications), and (c) SEQ ID NO: 12 (or SEQ ID NO: 12 with no more than three, two, or one amino acid modifications).

In some cases, an alpha chain polypeptide of a TCR provided herein can include the amino acid sequence set forth in SEQ ID NO:18 (or a variant of SEQ ID NO:18 with one, two, three, four, five, six, seven, eight, nine, or 10 amino acid modifications, provided that the alpha chain includes the appropriate combination of CDRs (or variants of the CDRs) described herein) or SEQ ID NO:22 (or a variant of SEQ ID NO:22 with one, two, three, four, five, six, seven, eight, nine, or 10 amino acid modifications, provided that the alpha chain includes the appropriate combination of CDRs (or variants of the CDRs) described herein). Amino acid modifications can be amino acid substitutions, amino acid deletions, or amino acid additions. Amino acid substitutions for SEQ ID NO:18 or SEQ ID NO:22 can be conservative amino acid substitutions or non-conservative amino acid substitutions as discussed above for SEQ ID NOs:1, 2, 3, 10, 11, 12, 23, 24, 25, 32, 33, and 34. In some cases, a variant of SEQ ID NO:18 or SEQ ID NO:22 can include no more than one, two, three, four, or five amino acid modifications.

In some cases, the constant region of a beta chain polypeptide of a TCR provided herein can have an amino acid sequence having at least 80% identity to the amino acid sequence set forth in SEQ ID NO:8 or SEQ ID NO:19. For example, the constant region of a beta chain polypeptide of a TCR provided herein can have an amino acid sequence having at least 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:8 or SEQ ID NO:19.

In some cases, the variable region of a beta chain polypeptide of a TCR provided herein can have an amino acid sequence having at least 80% identity to the amino acid sequence set forth in SEQ ID NO:47, provided that the beta chain includes the appropriate combination of CDRs (or variants of the CDRs) described herein. For example, the variable region of a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:47, provided that it includes (a) SEQ ID NO:1 (or SEQ ID NO:1 with no more than three, two, or one amino acid modifications), (b) SEQ ID NO:2 (or SEQ ID NO:2 with no more than three, two, or one amino acid modifications), and (c) SEQ ID NO:3 (or SEQ ID NO:3 with no more than three, two, or one amino acid modifications).

In some cases, the variable region of a beta chain polypeptide of a TCR provided herein can include the amino acid sequence set forth in SEQ ID NO:47 (or a variant of SEQ ID NO:47 with one, two, three, four, five, six, seven, eight, nine, or 10 amino acid modifications, provided that the beta chain includes the appropriate combination of CDRs (or variants of the CDRs) described herein). Amino acid modifications can be amino acid substitutions, amino acid deletions, or amino acid additions. Amino acid substitutions for SEQ ID NO:47 can be conservative amino acid substitutions or non-conservative amino acid substitutions as discussed above for SEQ ID NOs:1, 2, 3, 10, 11, 12, 23, 24, 25, 32, 33, and 34. In some cases, a variant of SEQ ID NO:47 can include no more than one, two, three, four, or five amino acid modifications.

In some cases, a beta chain polypeptide of a TCR provided herein can have an amino acid sequence having at least 80% identity to the amino acid sequence set forth in SEQ ID NO:9 or SEQ ID NO:20, provided that the beta chain includes the appropriate combination of CDRs (or variants of the CDRs) described herein. For example, a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:9, provided that it includes (a) SEQ ID NO:1 (or SEQ ID NO:1 with no more than three, two, or one amino acid modifications), (b) SEQ ID NO:2 (or SEQ ID NO:2 with no more than three, two, or one amino acid modifications), and (c) SEQ ID NO:3 (or SEQ ID NO:3 with no more than three, two, or one amino acid modifications). In another example, a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:20, provided that it includes (a) SEQ ID NO:1 (or SEQ ID NO:1 with no more than three, two, or one amino acid modifications), (b) SEQ ID NO:2 (or SEQ ID NO:2 with no more than three, two, or one amino acid modifications), and (c) SEQ ID NO:3 (or SEQ ID NO:3 with no more than three, two, or one amino acid modifications).

In some cases, a beta chain polypeptide of a TCR provided herein can have an amino acid sequence having at least 80% identity to the amino acid sequence set forth the amino acid sequence set forth in SEQ ID NO:9 (or a variant of SEQ ID NO:9 with one, two, three, four, five, six, seven, eight, nine, or 10 amino acid modifications, provided that the beta chain includes the appropriate combination of CDRs (or variants of the CDRs) described herein) or SEQ ID NO:20 (or a variant of SEQ ID NO:20 with one, two, three, four, five, six, seven, eight, nine, or 10 amino acid modifications, provided that the beta chain includes the appropriate combination of CDRs (or variants of the CDRs) described herein). Amino acid modifications can be amino acid substitutions, amino acid deletions, or amino acid additions. Amino acid substitutions for SEQ ID NO:9 or SEQ ID NO:20 can be conservative amino acid substitutions or non-conservative amino acid substitutions as discussed above for SEQ ID NOs:1, 2, 3, 10, 11, 12, 23, 24, 25, 32, 33, and 34. In some cases, a variant of SEQ ID NO:9 or SEQ ID NO:20 can include no more than one, two, three, four, or five amino acid modifications.

In some cases, the constant region of an alpha chain polypeptide of a TCR provided herein can have an amino acid sequence having at least 80% identity to the amino acid sequence set forth in SEQ ID NO:39 or SEQ ID NO:43. For example, the constant region of an alpha chain polypeptide of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:39 or SEQ ID NO:43.

In some cases, the variable region of an alpha chain polypeptide of a TCR provided herein can have an amino acid sequence having at least 80% identity to the amino acid sequence set forth in SEQ ID NO:46, provided that the alpha chain includes the appropriate combination of CDRs (or variants of the CDRs) described herein. For example, the variable region of an alpha chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:46, provided that it includes (a) SEQ ID NO:32 (or SEQ ID NO:32 with no more than three, two, or one amino acid modifications), (b) SEQ ID NO:33 (or SEQ ID NO:33 with no more than three, two, or one amino acid modifications), and (c) SEQ ID NO:34 (or SEQ ID NO:34 with no more than three, two, or one amino acid modifications).

In some cases, the variable region of an alpha chain polypeptide of a TCR provided herein can include the amino acid sequence set forth in SEQ ID NO:46 (or a variant of SEQ ID NO:46 with one, two, three, four, five, six, seven, eight, nine, or 10 amino acid modifications, provided that the alpha chain includes the appropriate combination of CDRs (or variants of the CDRs) described herein). Amino acid modifications can be amino acid substitutions, amino acid deletions, or amino acid additions. Amino acid substitutions for SEQ ID NO:46 can be conservative amino acid substitutions or non-conservative amino acid substitutions as discussed above for SEQ ID NOs:1, 2, 3, 10, 11, 12, 23, 24, 25, 32, 33, and 34. In some cases, a variant of SEQ ID NO:46 can include no more than one, two, three, four, or five amino acid modifications.

In some cases, an alpha chain polypeptide of a TCR provided herein can have an amino acid sequence having at least 80% identity to the amino acid sequence set forth in SEQ ID NO:40 or SEQ ID NO:44, provided that the alpha chain includes the appropriate combination of CDRs (or variants of the CDRs) described herein. For example, an alpha chain of a TCR provided herein can have an amino acid sequence having at least 80%, 82%, 83%, 84%, 85%, 86%, 87%, 88%, 89%, 90%9, 91%, 92%, 93%, 94%, 95%, 96%, 97%, 98%, 99%, or 100% identity to the amino acid sequence set forth in SEQ ID NO:40, provided that it includes (a) SEQ ID NO:32 (or SEQ ID NO:32 with no more than three, two, or one amino acid modifications), (b) SEQ ID NO:33 (or SEQ ID NO:33 with no more than three, two, or one amino acid modifications), and (c) SEQ ID NO:34 (or SEQ ID NO:34 with no more than three, two, or one amino acid modifications). In another example, an alpha chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:44, provided that it includes (a) SEQ ID NO:32 (or SEQ ID NO:32 with no more than three, two, or one amino acid modifications), (b) SEQ ID NO:33 (or SEQ ID NO:33 with no more than three, two, or one amino acid modifications), and (c) SEQ ID NO: 34 (or SEQ ID NO:34 with no more than three, two, or one amino acid modifications).

In some cases, an alpha chain polypeptide of a TCR provided herein can include the amino acid sequence set forth in SEQ ID NO:40 (or a variant of SEQ ID NO:40 with one, two, three, four, five, six, seven, eight, nine, or 10 amino acid modifications, provided that the alpha chain includes the appropriate combination of CDRs (or variants of the CDRs) described herein) or SEQ ID NO:44 (or a variant of SEQ ID NO:44 with one, two, three, four, five, six, seven, eight, nine, or 10 amino acid modifications, provided that the alpha chain includes the appropriate combination of CDRs (or variants of the CDRs) described herein). Amino acid modifications can be amino acid substitutions, amino acid deletions, or amino acid additions. Amino acid substitutions for SEQ ID NO:40 or SEQ ID NO:44 can be conservative amino acid substitutions or non-conservative amino acid substitutions as discussed above for SEQ ID NOs:1, 2, 3, 10, 11, 12, 23, 24, 25, 32, 33, and 34. In some cases, a variant of SEQ ID NO:40 or SEQ ID NO:44 can include no more than one, two, three, four, or five amino acid modifications.

In some cases, the constant region of a beta chain polypeptide of a TCR provided herein can have an amino acid sequence having at least 80% identity to the amino acid sequence set forth in SEQ ID NO:30 or SEQ ID NO:41. For example, the constant region of a beta chain polypeptide of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:30 or SEQ ID NO:41.

In some cases, the variable region of a beta chain polypeptide of a TCR provided herein can have an amino acid sequence having at least 80% identity to the amino acid sequence set forth in SEQ ID NO:48, provided that the beta chain includes the appropriate combination of CDRs (or variants of the CDRs) described herein. For example, the variable region of a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:48, provided that it includes (a) SEQ ID NO:23 (or SEQ ID NO:23 with no more than three, two, or one amino acid modifications), (b) SEQ ID NO:24 (or SEQ ID NO:24 with no more than three, two, or one amino acid modifications), and (c) SEQ ID NO:25 (or SEQ ID NO:25 with no more than three, two, or one amino acid modifications).

In some cases, the variable region of a beta chain polypeptide of a TCR provided herein can include the amino acid sequence set forth in SEQ ID NO:48 (or a variant of SEQ ID NO:48 with one, two, three, four, five, six, seven, eight, nine, or 10 amino acid modifications, provided that the beta chain includes the appropriate combination of CDRs (or variants of the CDRs) described herein). Amino acid modifications can be amino acid substitutions, amino acid deletions, or amino acid additions. Amino acid substitutions for SEQ ID NO:48 can be conservative amino acid substitutions or non-conservative amino acid substitutions as discussed above for SEQ ID NOs:1, 2, 3, 10, 11, 12, 23, 24, 25, 32, 33, and 34. In some cases, a variant of SEQ ID NO:48 can include no more than one, two, three, four, or five amino acid modifications.

In some cases, a beta chain polypeptide of a TCR provided herein can have an amino acid sequence having at least 80% identity to the amino acid sequence set forth in SEQ ID NO:31 or SEQ ID NO:42, provided that the beta chain includes the appropriate combination of CDRs (or variants of the CDRs) described herein. For example, a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:31, provided that it includes (a) SEQ ID NO:23 (or SEQ ID NO:23 with no more than three, two, or one amino acid modifications), (b) SEQ ID NO:24 (or SEQ ID NO:24 with no more than three, two, or one amino acid modifications), and (c) SEQ ID NO:25 (or SEQ ID NO:25 with no more than three, two, or one amino acid modifications). In another example, a beta chain of a TCR provided herein can have an amino acid sequence having at least 80%, 82%, 83%, 84%, 85%, 86%, 87%, 88%, 89%, 90%, 91%, 92%, 93%, 94%, 95%, 96%, 97%, 98%, 99%, or 100% identity to the amino acid sequence set forth in SEQ ID NO:42, provided that it includes (a) SEQ ID NO:23 (or SEQ ID NO:23 with no more than three, two, or one amino acid modifications), (b) SEQ ID NO:24 (or SEQ ID NO:24 with no more than three, two, or one amino acid modifications), and (c) SEQ ID NO:25 (or SEQ ID NO:25 with no more than three, two, or one amino acid modifications).

In some cases, a beta chain polypeptide of a TCR provided herein can have an amino acid sequence having at least 80% identity to the amino acid sequence set forth the amino acid sequence set forth in SEQ ID NO:31 (or a variant of SEQ ID NO:31 with one, two, three, four, five, six, seven, eight, nine, or 10 amino acid modifications, provided that the beta chain includes the appropriate combination of CDRs (or variants of the CDRs) described herein) or SEQ ID NO:42 (or a variant of SEQ ID NO:42 with one, two, three, four, five, six, seven, eight, nine, or 10 amino acid modifications, provided that the beta chain includes the appropriate combination of CDRs (or variants of the CDRs) described herein). Amino acid modifications can be amino acid substitutions, amino acid deletions, or amino acid additions. Amino acid substitutions for SEQ ID NO:31 or SEQ ID NO:42 can be conservative amino acid substitutions or non-conservative amino acid substitutions as discussed above for SEQ ID NOs:1, 2, 3, 10, 11, 12, 23, 24, 25, 32, 33, and 34. In some cases, a variant of SEQ ID NO:31 or SEQ ID NO:42 can include no more than one, two, three, four, or five amino acid modifications.

In some cases, a TCR provided herein can include no more than four (e.g., four, three, two, or one) amino acid modifications (e.g., amino acid substitutions, amino acid deletions, or amino acid additions) in one, two, three, or all four of the framework regions (the regions between the CDRs). For example, a TCR provided herein that has affinity for a SF3B1 peptide can include no more than four (e.g., four, three, two, or one) amino acid modifications in one, two, three, or all four of the alpha chain framework regions set forth in SEQ ID NOs:13, 14, 15, and 16 and/or the beta chain framework regions set forth in SEQ ID NOs:4, 5, 6, and 7. For example, a TCR provided herein that has affinity for a SF3B1 peptide can include no more than four (e.g., four, three, two, or one) amino acid modifications in one, two, three, or all four of the alpha chain framework regions set forth in SEQ ID NOs:35, 36, 37, and 38 and/or the beta chain framework regions set forth in SEQ ID NOs:26, 27, 28, and 29. In some cases, a TCR provided herein can include no more than four (e.g., four, three, two, or one) amino acid modifications (e.g., amino acid substitutions, amino acid deletions, or amino acid additions) in one, two, three, or all four of the framework regions set forth in SEQ ID NOs:4, 5, 6, 7, 13, 14, 15, and 16 and (i) one, two, or three amino acid modifications in one, two, or all three CDRs of the alpha chain variable region having the amino acid sequences set forth in SEQ ID NOs:10, 11, and 12 and/or (ii) one, two, or three amino acid modifications in one, two, or all three CDRs of the beta chain variable region having the amino acid sequences set forth in SEQ ID NOs:1, 2, and 3. In some cases, a TCR provided herein can include no more than four (e.g., four, three, two, or one) amino acid modifications (e.g., amino acid substitutions, amino acid deletions, or amino acid additions) in one, two, three, or all four of the framework regions set forth in SEQ ID NOs:26, 27, 28, 29, 35, 36, 37, and 38 and (i) one, two, or three amino acid modifications in one, two, or all three CDRs of the alpha chain variable region having the amino acid sequences set forth in SEQ ID NOs:32, 33, and 34 and/or (ii) one, two, or three amino acid modifications in one, two, or all three CDRs of the beta chain variable region having the amino acid sequences set forth in SEQ ID NOs:23, 24, and 25.

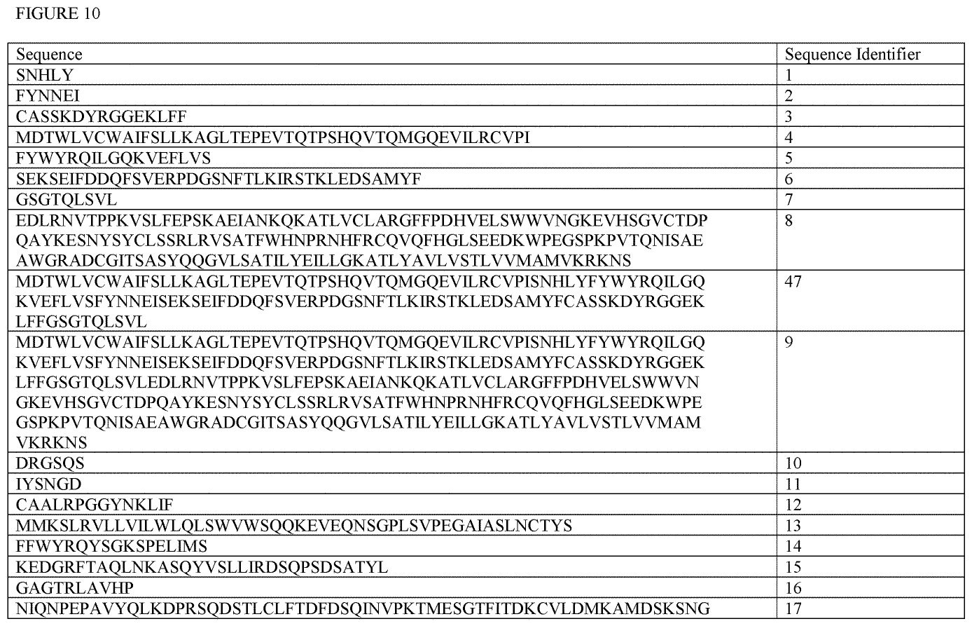

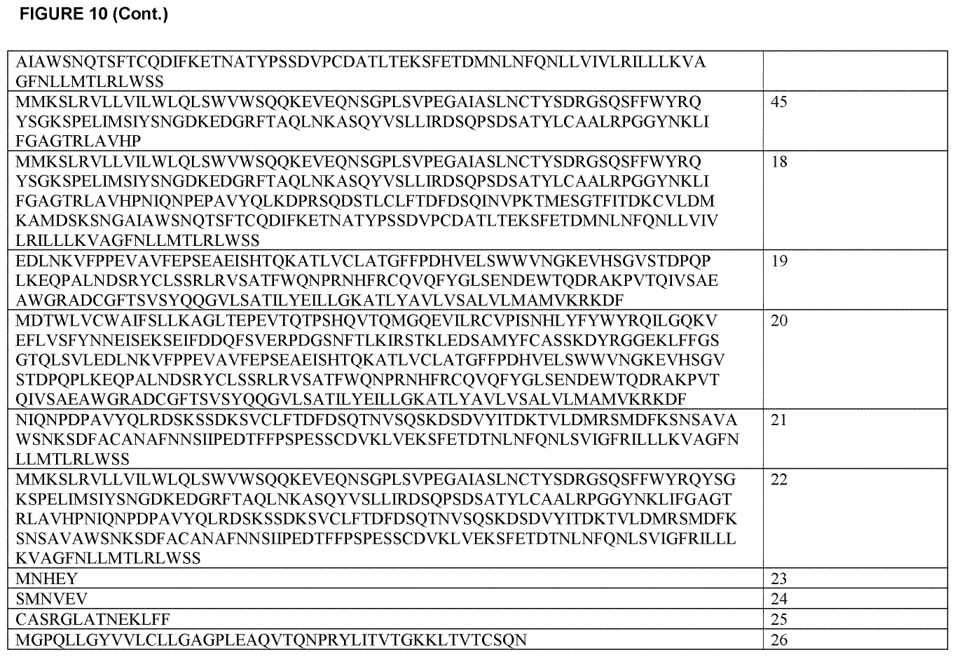

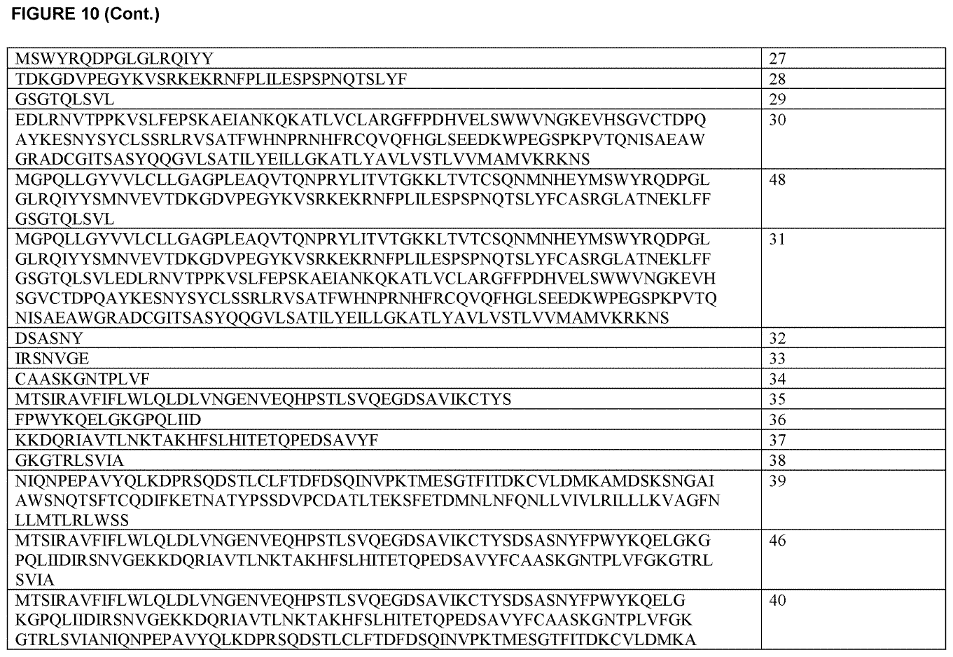

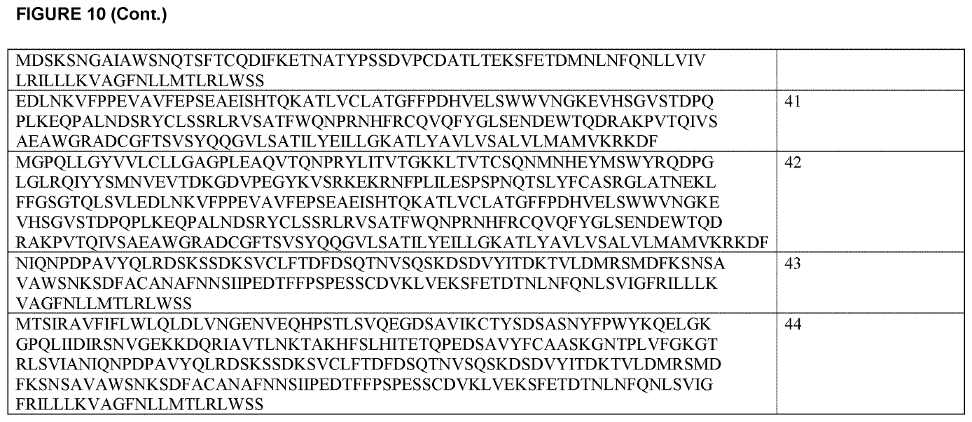

In some cases, a TCR provided herein can have the sequences as set forth in Table 2 (see FIG. 10 for the sequences).

TABLE 2
Exemplary TCRs.

| | | | SEQ ID | | | | | SEQ ID | | |
|---|---|---|---|---|---|---|---|---|---|---|
| | | | of | | | | SEQ ID NO | of | | |
| | SEQ ID | SEQ ID NO | constant | SEQ ID | SEQ | | of alpha | Constant | SEQ ID | SEQ ID |
| | NO of | of beta chain | region | NO of | ID NO | SEQ ID NOs | chain | region in | NO of | NO of |
| | beta chain | Framework | in beta | variable | of beta | of alpha | Framework | alpha | variable | alpha |
| TCR | CDRs | regions | chain | region | chain | chain CDRs | regions | chain | region | chain |
| TRBV2-01 + | 1, 2, 3 | 4, 5, 6, 7 | 8 | 47 | 9 | 10, 11, 12 | 13, 14, 15, 16 | 17 | 45 | 18 |
| TRAV12-02 | | | | | | | | | | |
| Chimeric TCR | | | | | | | | | | |
| TRBV2-01 + | 1, 2, 3 | 4, 5, 6, 7 | 19 | 47 | 20 | 10, 11, 12 | 13, 14, 15, 16 | 21 | 45 | 22 |
| TRAV12-02 | | | | | | | | | | |
| Human TCR | | | | | | | | | | |
| TRBV27-01 + | 23, 24, 25 | 26, 27, 28, 29 | 30 | 48 | 31 | 32, 33, 34 | 35, 36, 37, 38 | 39 | 46 | 40 |
| TRAV13-01 | | | | | | | | | | |
| Chimeric TCR | | | | | | | | | | |
| TRBV27-01 + | 23, 24, 25 | 26, 27, 28, 29 | 41 | 48 | 42 | 32, 33, 34 | 35, 36, 37, 38 | 43 | 46 | 44 |
| TRAV13-01 | | | | | | | | | | |
| Human TCR | | | | | | | | | | |

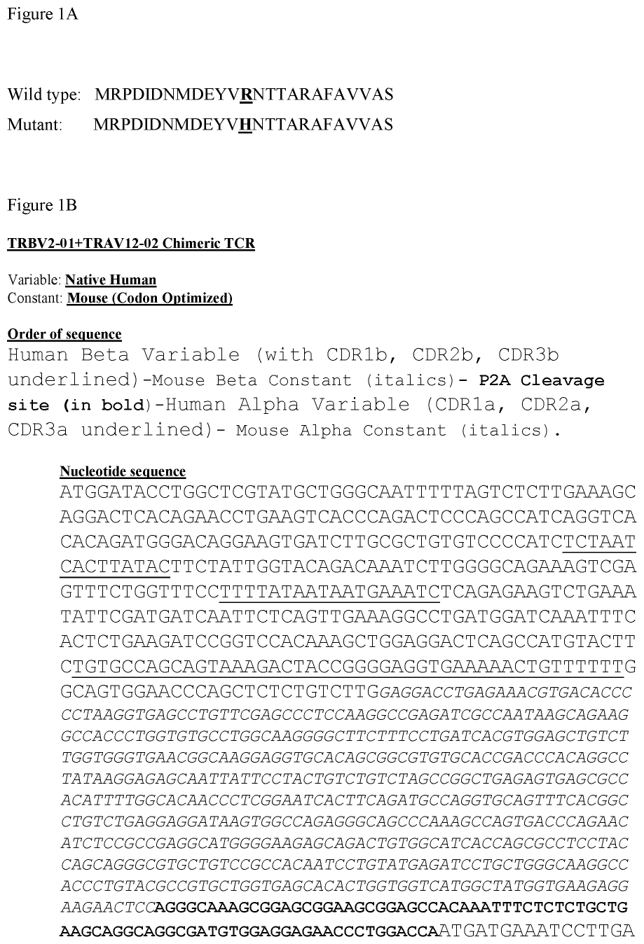

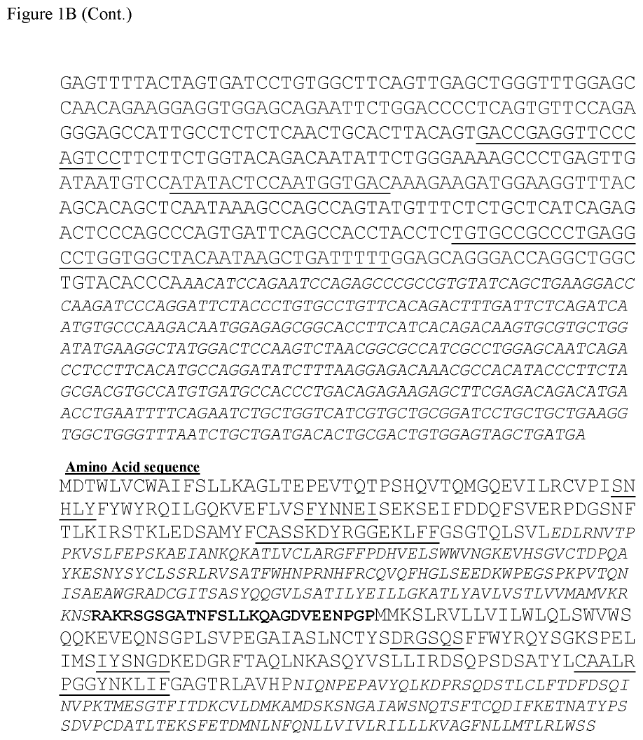

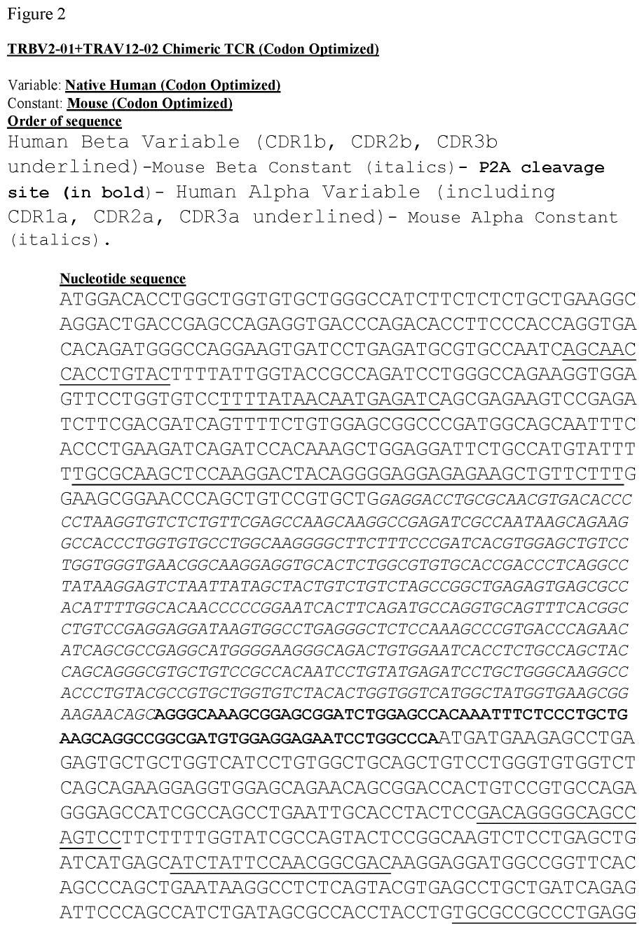

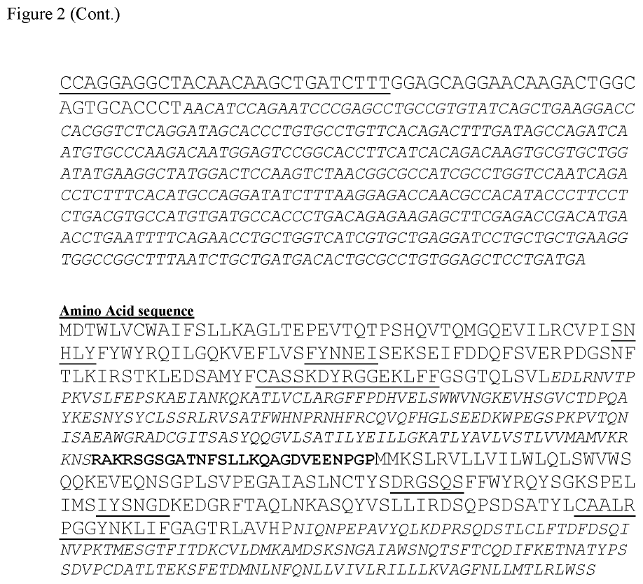

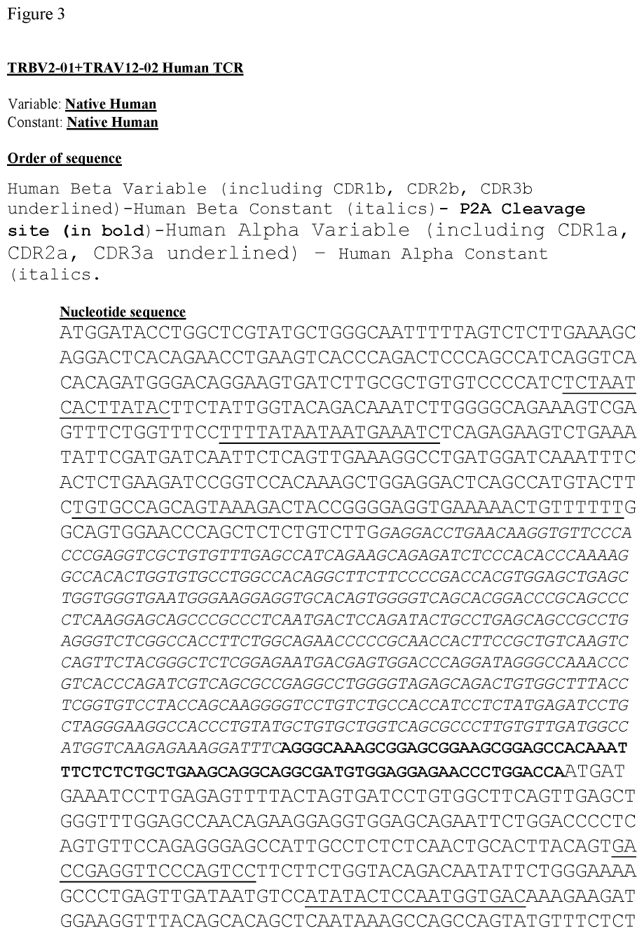

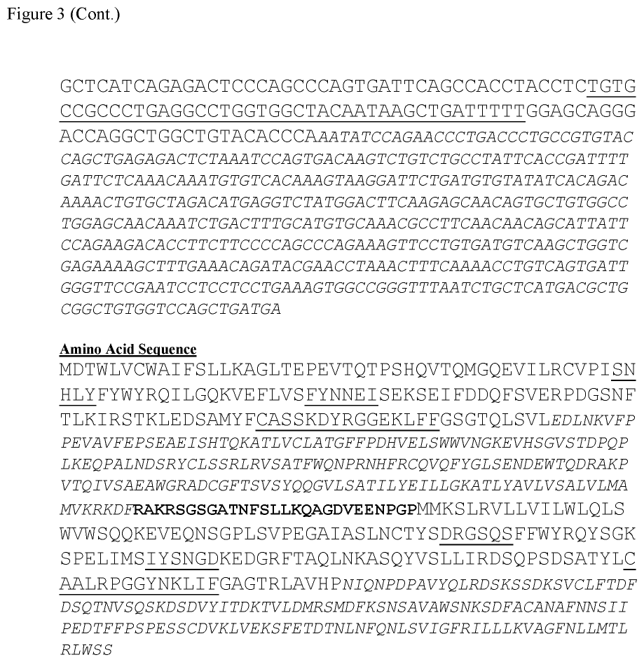

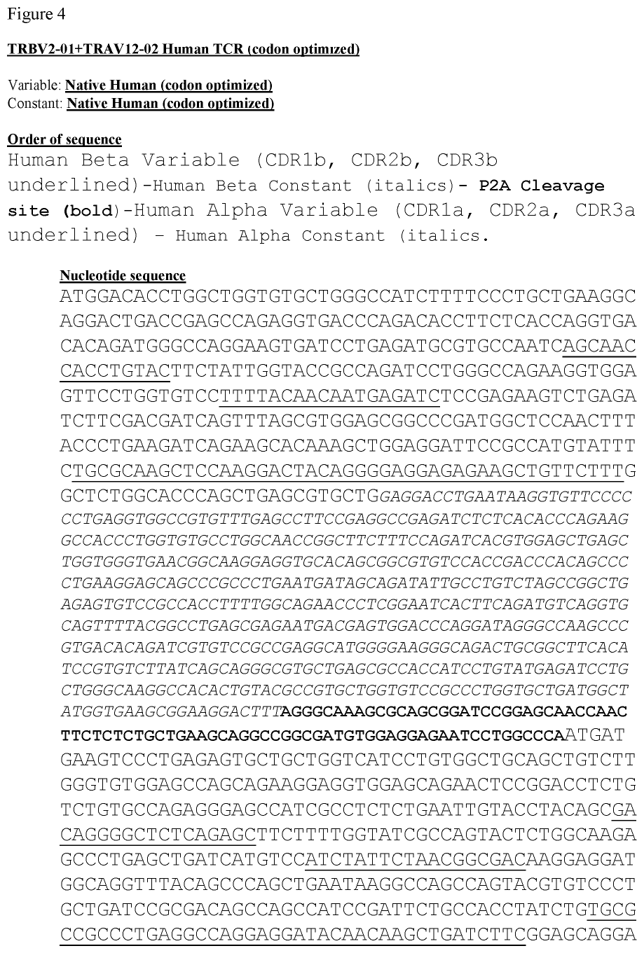

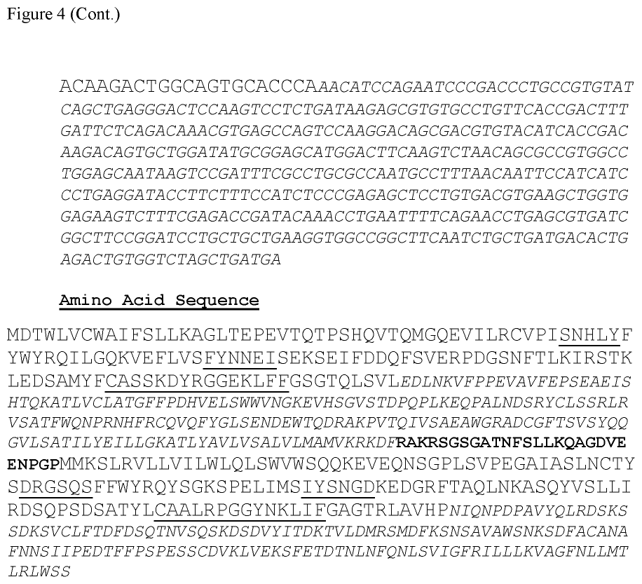

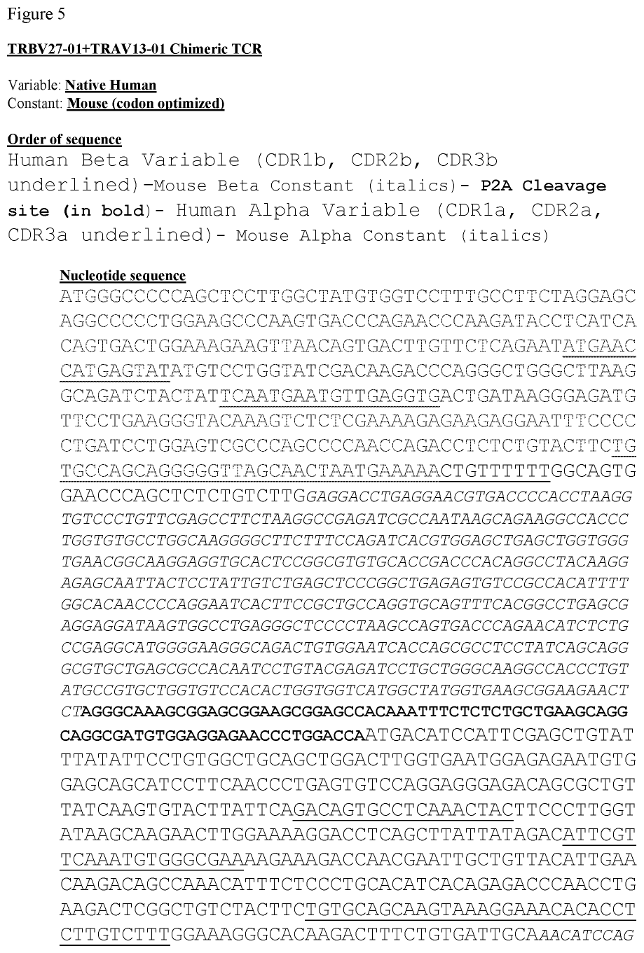

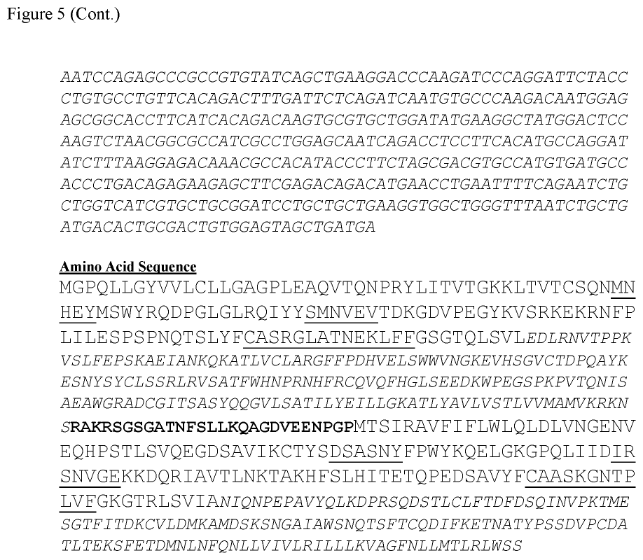

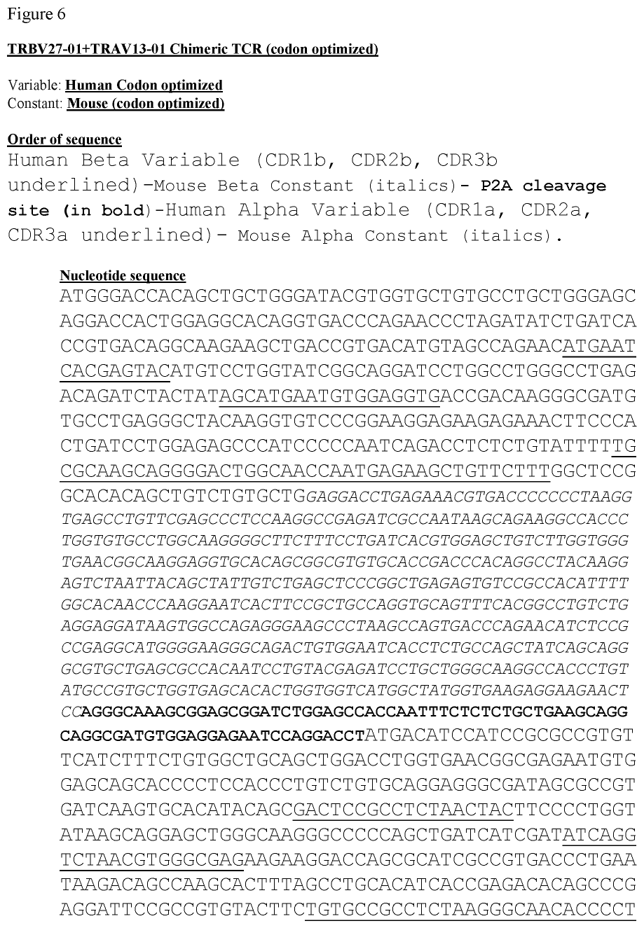

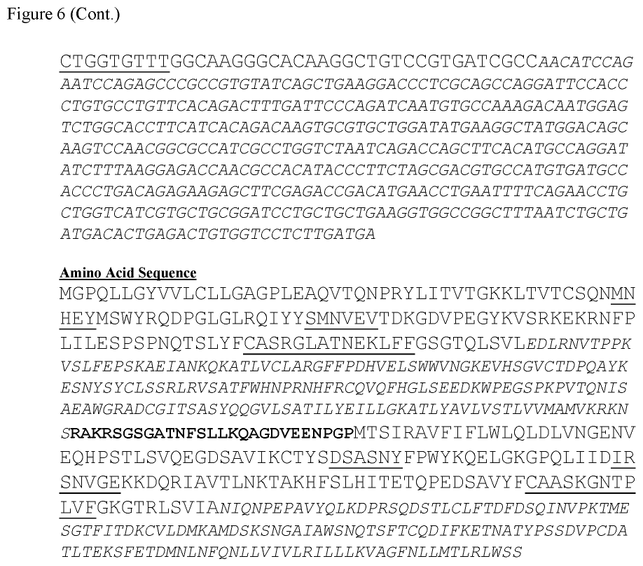

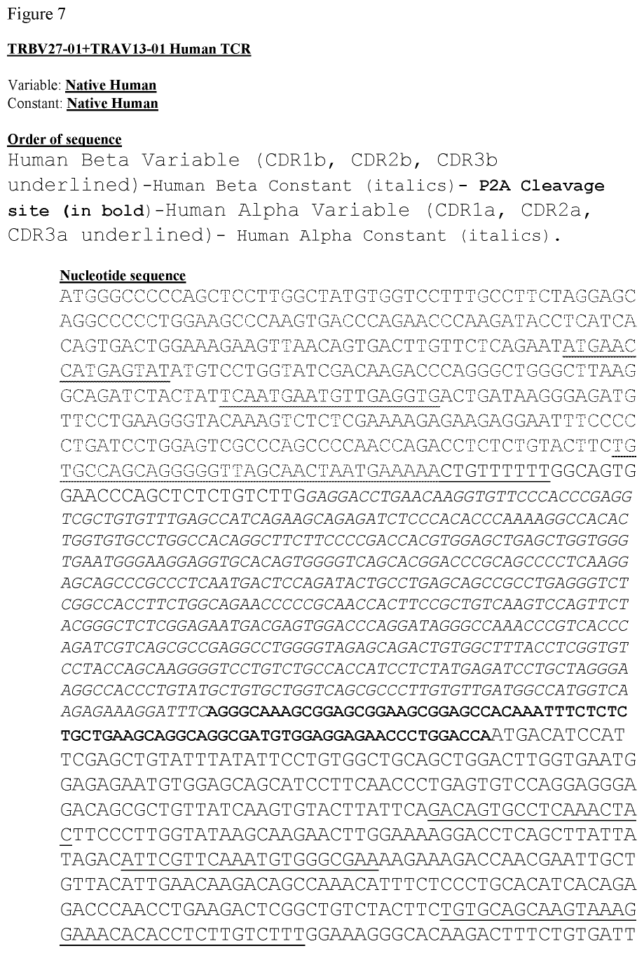

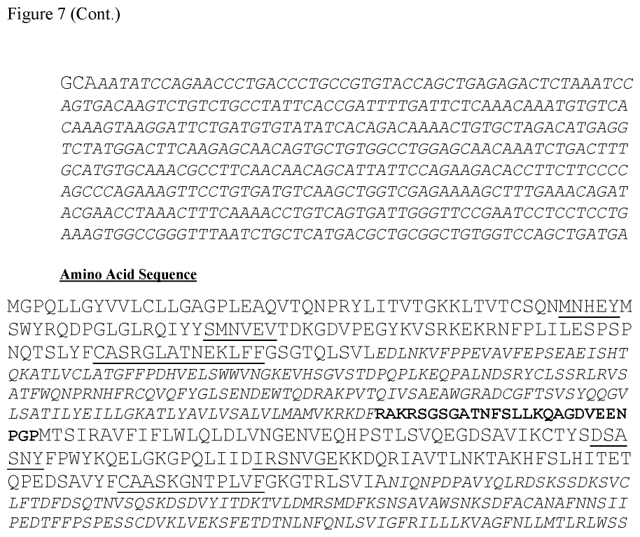

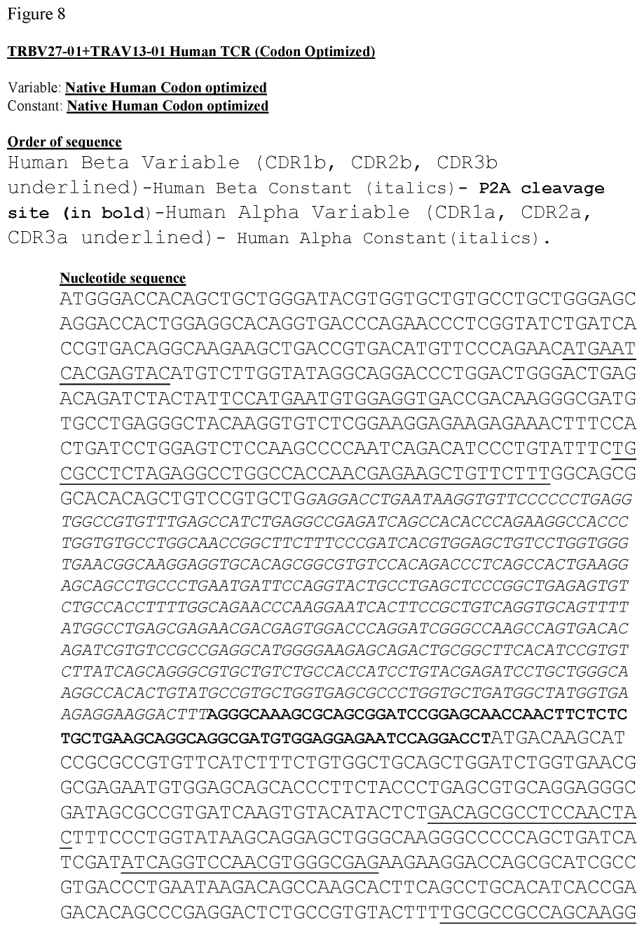

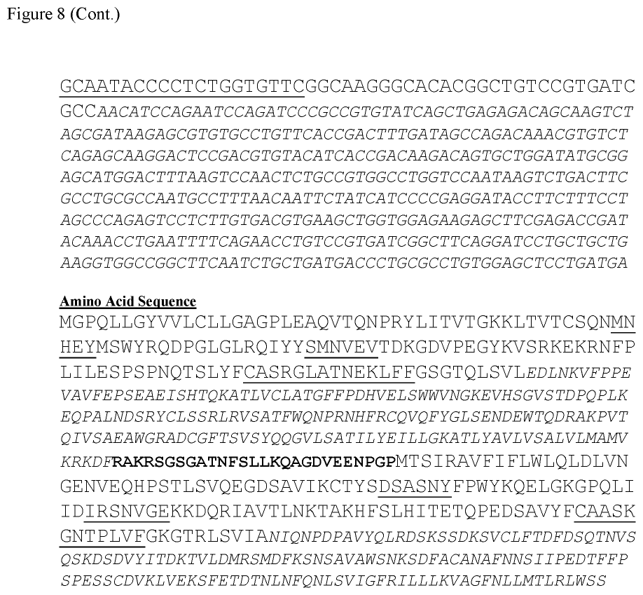

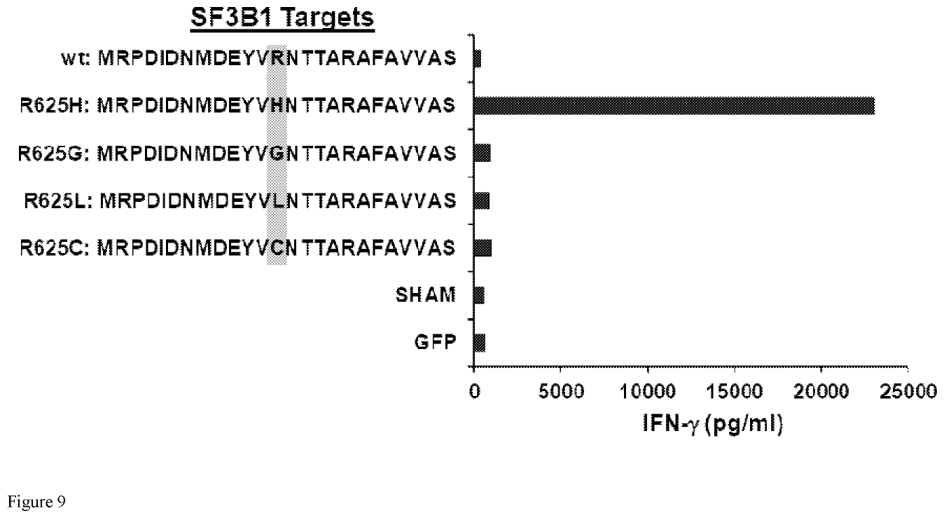

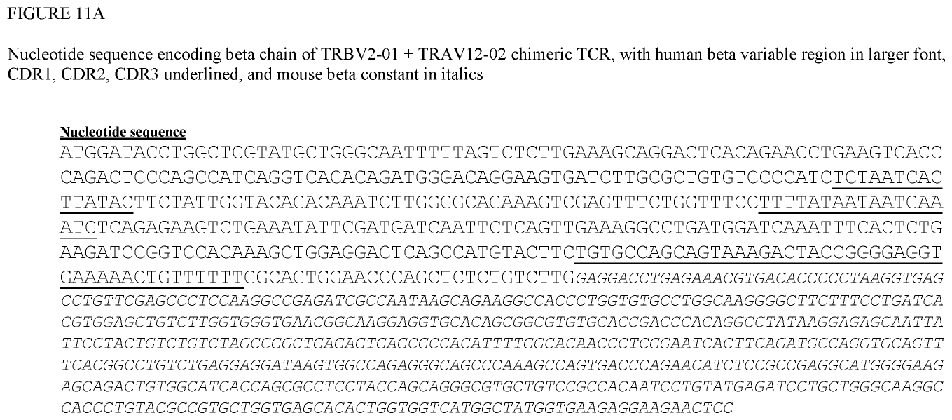

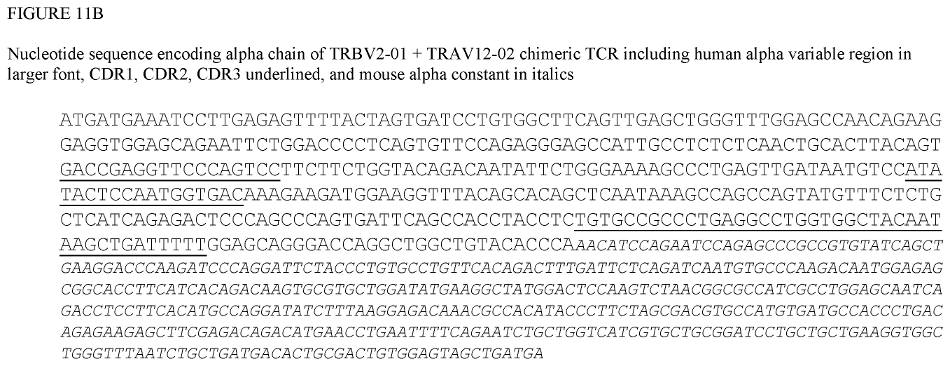

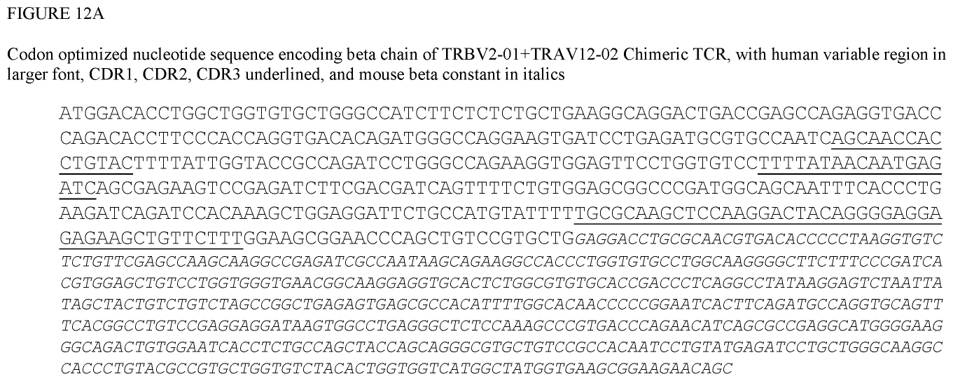

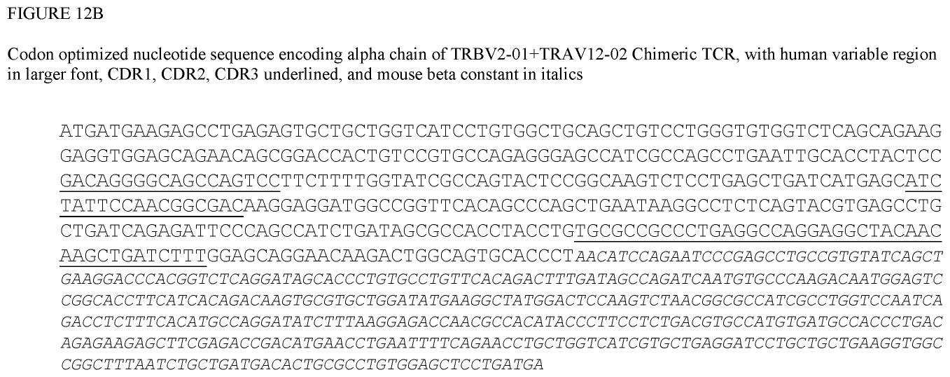

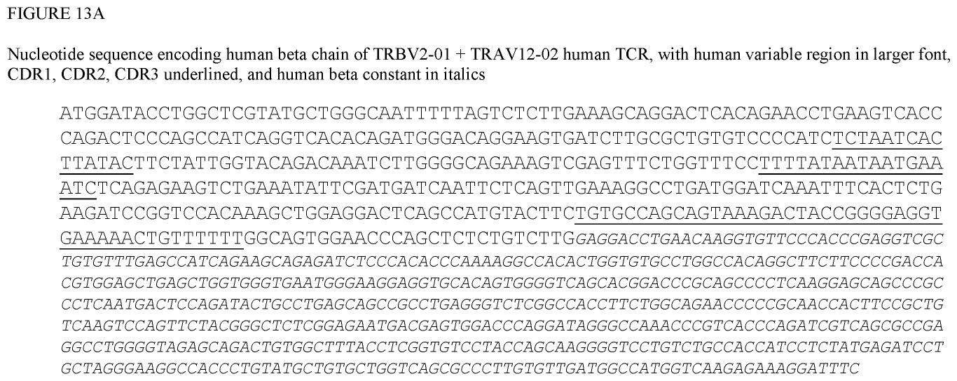

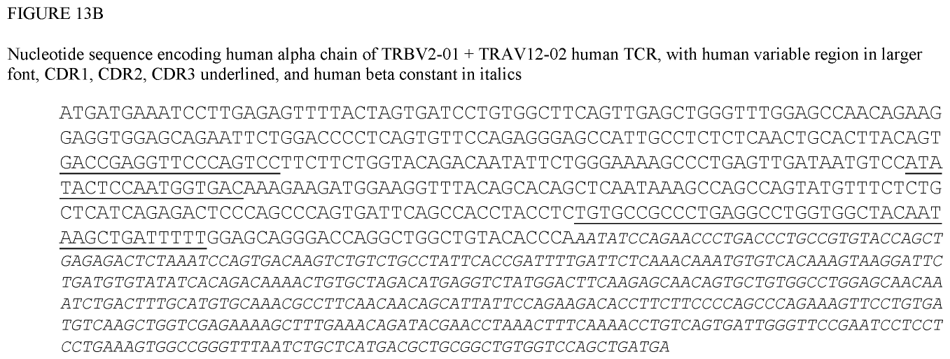

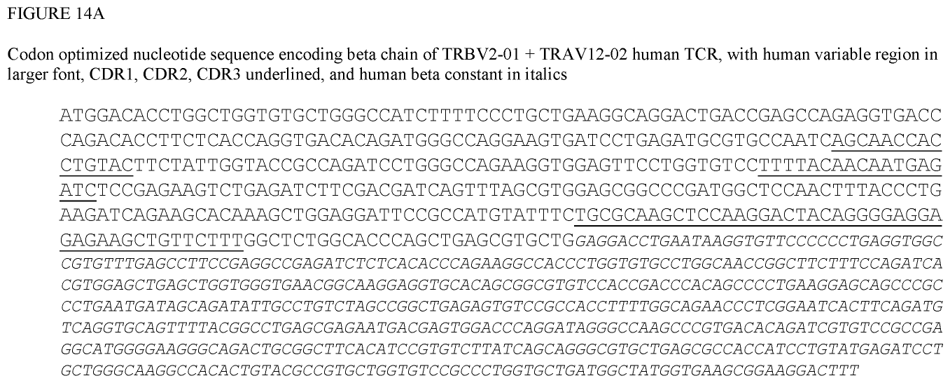

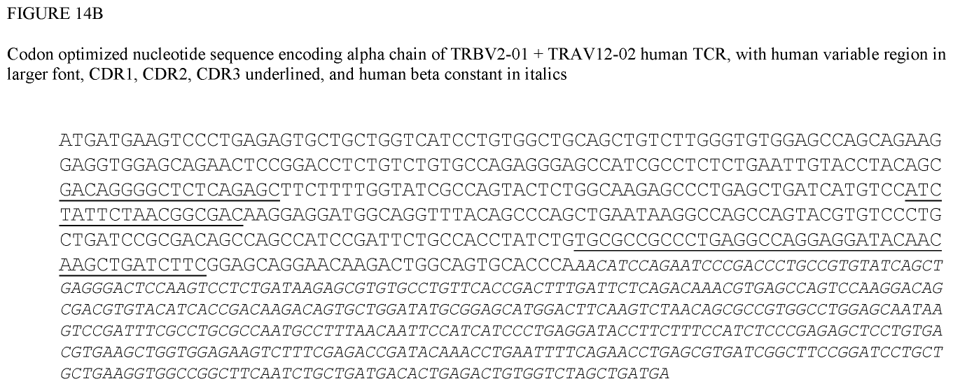

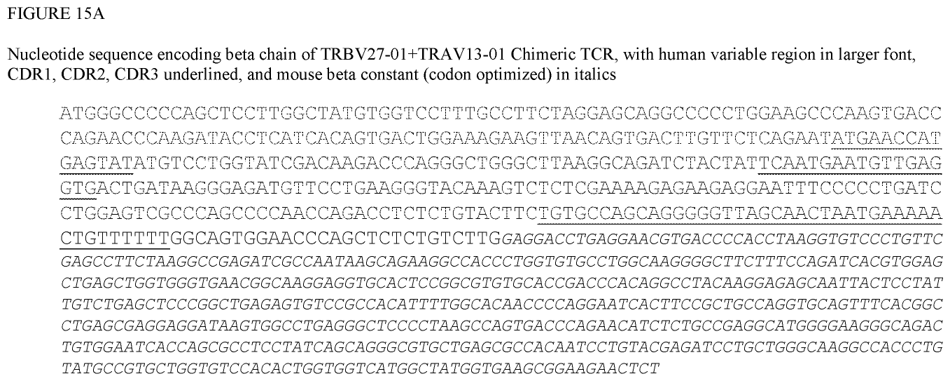

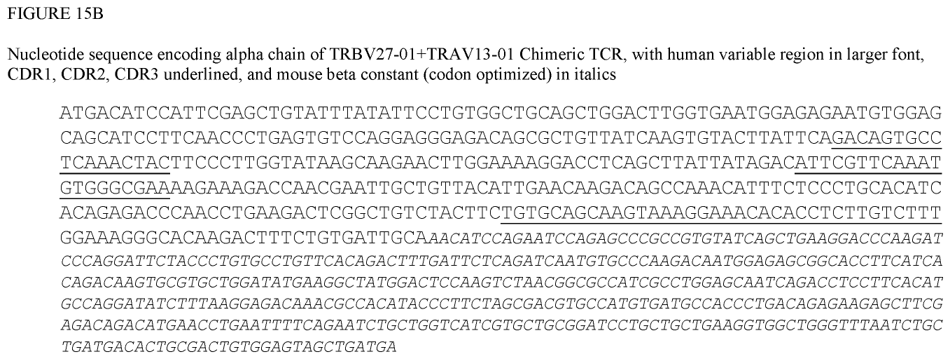

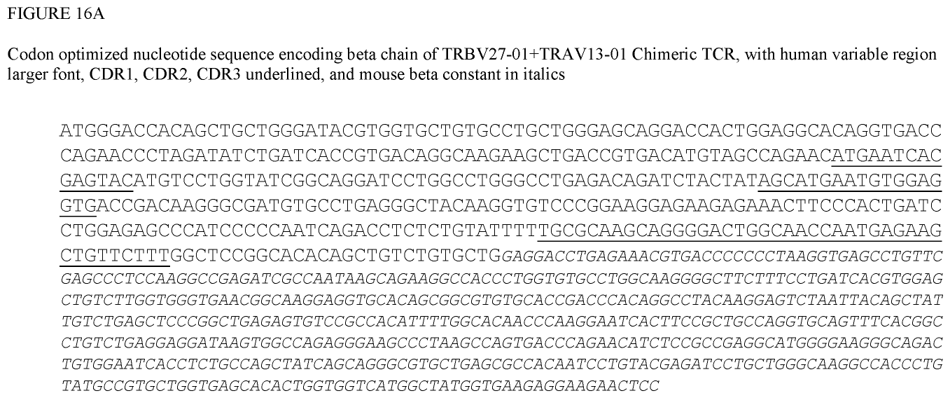

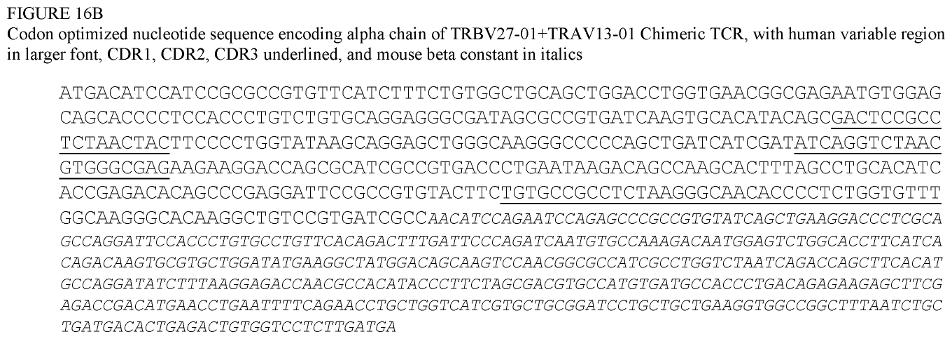

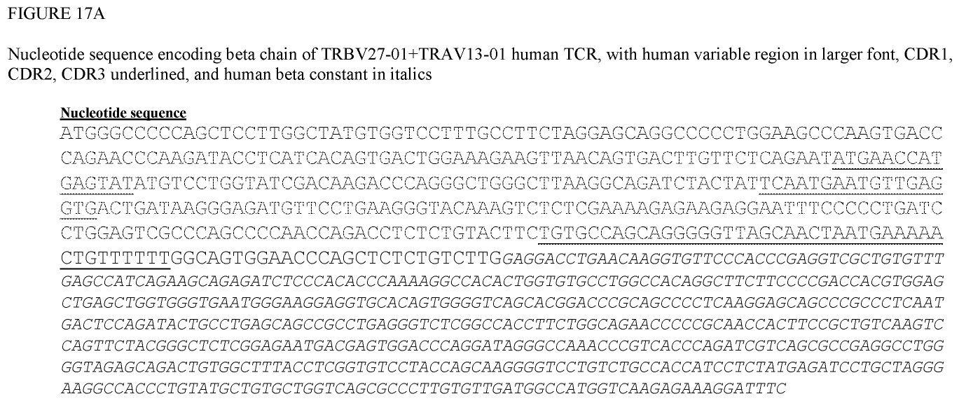

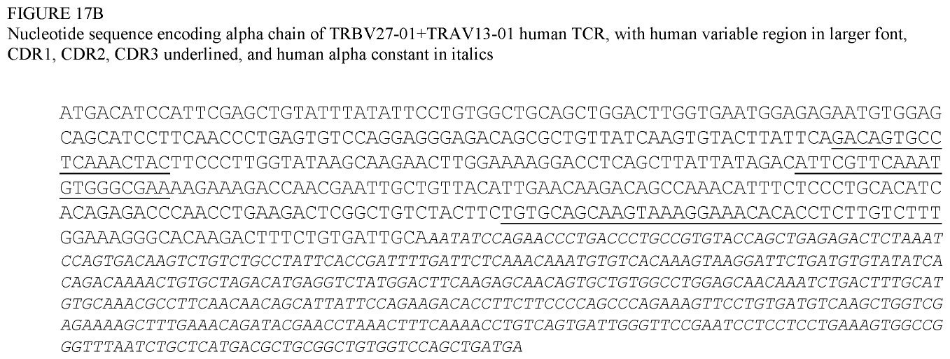

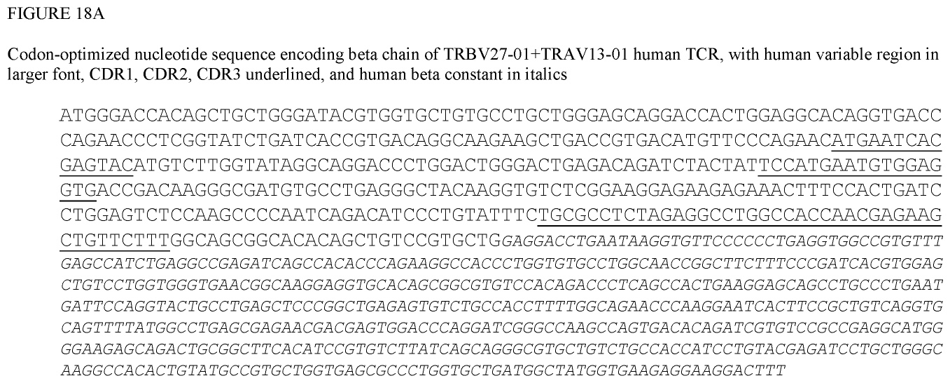

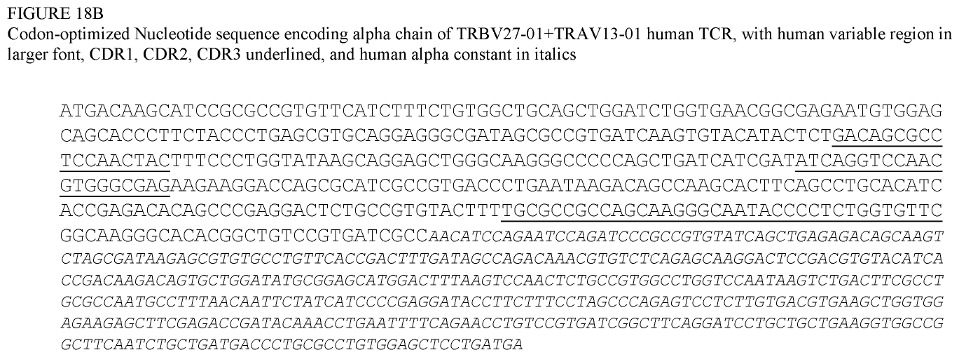

This document also provides isolated nucleic acids that encode an alpha and/or beta chain polypeptide of a TCR provided herein. See, for example, FIG. 1B, FIG. 3, FIG. 5, FIG. 9, FIG. 11A, FIG. 11B, FIG. 13A, FIG. 13B, FIG. 15A, FIG. 15B, FIG. 17A, or FIG. 17B. In some cases, the nucleic acid sequence can be codon optimized for expression in a host cell (e.g., a human host cell). See, for example, FIG. 2, FIG. 4, FIG. 6, FIG. 8, FIG. 12A, FIG. 12B, FIG. 14A, FIG. 14B, FIG. 16A, FIG. 16B, FIG. 18A, or FIG. 18B. In addition, this document provides vectors containing one or more of such nucleic acids. The nucleic acids provided herein can be single stranded and double stranded nucleic acids of any appropriate type (e.g., DNA, RNA, or DNA/RNA hybrids). The nucleic acids provided herein can be used therapeutically or can be used in methods for producing a TCR.

In some cases, a nucleic acid provided herein includes a nucleic acid sequence that encodes an alpha chain of a TCR and a nucleic acid sequence that encodes a beta chain of a TCR. The nucleic acid also can include a nucleic acid sequence encoding a linker. The nucleic acid sequence encoding the linker can be between the nucleic acid encoding an alpha chain and the nucleic acid encoding the beta chain to allow the alpha and beta chains to be encoded by the same contiguous nucleic acid sequence. For example, the nucleic acid sequence encoding the linker can be 3′ of the nucleic acid sequence encoding the alpha chain and 5′ of the nucleic acid sequence encoding the beta chain or it can be 3′ of the nucleic acid sequence encoding the beta chain and 5′ of the nucleic acid sequence encoding the alpha chain.

In some cases, the linker can be a self-cleaving peptide (e.g., a 2A peptide) such that during translation of the transcripts, the growing polypeptide can be cleaved at the 2A peptide with translation continuing through to the next chain. When designing a vector to express the alpha and beta chains as a multicistronic unit, the nucleic acid encoding the alpha and beta chains and the self-cleaving peptide (e.g., a 2A peptide) can be designed such that they are in translational frame with each other. Examples of 2A peptides that can be used as described herein include, without limitation, a 2A peptide of foot-and-mouth disease virus (FMDV), a 2A peptide of equine rhinitis A virus (ERAVO), a 2A peptide of Thosea asigna virus (TaV), or a 2A peptide of porcine teschovirus-1 (PTV-1). 2A peptides derived from FMDV, ERAV, PTV-1, and TaV are referred to herein as “F2A,” “E2A,” “P2A,” and “T2A,” respectively. Table 3 provides exemplary amino acid sequences of 2A peptides.

TABLE 3
Exemplary 2A peptides that can be used as
described herein.

| Type | Peptide Sequence | SEQ ID NO: |
|---|---|---|
| P2A | GSGATNFSLLKQAGDVEENPGP | 80 |
| | or | |
| | RAKRSGSGATNFSLLKQAGDVEENPGP | 81 |
| T2A | GSGEGRGSLLTCGDVEENPGP | 82 |
| E2A | GSGQCTNYALLKLAGDVESNPGP | 83 |
| F2A | GSGVKQTLNFDLLKLAGDVESNPGP | 84 |

In some cases, an Internal Ribosome Entry Site (IRES) can be used in place of (or in addition to) a self-cleaving peptide. Examples of IRES sequences that can be used as described herein include, without limitation, an Encephalomyocrditis virus (EMCV) IRES (e.g., IRES2), a Hepatitis C virus (HCV) IRES, a Picoma virus IRES, and a Pestivirus IRES.

In some cases, a linker can include a furin cleavage site. Furin is a ubiquitously expressed protease that resides in the trans-golgi and processes protein precursors before their secretion. Furin cleaves at the COOH terminus of its consensus recognition sequence. Examples of furin consensus recognition sequences (or “furin cleavage sites”) that can be used as described herein include, without limitation, Arg-X-Lys-Arg (SEQ ID NO:84) or Arg-X-Arg-Arg (SEQ ID NO:85), (Lys/Arg)-Arg-X-(Lys/Arg)-Arg (SEQ ID NO:86) and Arg-X-X-Arg (SEQ ID NO:87), such as an Arg-Gln-Lys-Arg (SEQ ID NO:88), where X is any naturally occurring amino acid.

A vector that includes one or more nucleic acids that encode an alpha chain and/or beta chain of a TCR provided herein can be a nucleic acid vector (e.g., naked DNA or plasmid vector) or a viral vector. Examples of viral vectors that can be designed to include one or more nucleic acids encoding an alpha chain and/or beta chain of a TCR provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), or poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2):176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids encoding an alpha chain and/or beta chain of a TCR provided herein. Any appropriate nucleic acid or viral vector construction methods can be used to make a nucleic acid vector that includes one or more nucleic acids encoding an alpha chain and/or beta chain of a TCR provided herein (see, e.g., Green and Sambrook, Molecular Cloning: A Laboratory Manual, 4th edition, Cold Spring Harbor Laboratory, NY (2012); and Ausubel et al., Current Protocols in Molecular Biology, Green Publishing Associates and John Wiley & Sons, New York, N.Y. (1987-2008)).

A vector provided herein (e.g., a nucleic acid or viral vector provided herein) can include any appropriate promoter and/or other regulatory sequences (e.g., transcription and translation initiation and termination codons) operably linked the nucleic acid encoding a polypeptide (e.g., an alpha chain and/or beta chain of a TCR provided herein). In some cases, a promoter used to drive expression of an alpha chain and/or beta chain of a TCR provided herein can be a constitutive or regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, and AAV promoters. In some embodiments, the promoter can be a viral 5′ long terminal repeat (LTR) or 3′ LTR.

In some cases, a vector includes a separate promoter to drive expression of each chain instead of using a linker between the chains. In these cases, one promoter sequence can drive expression of an alpha chain, and a separate promoter sequence can drive expression of a beta chain. These two promoter sequences can be the same or different.

This document also provides cells (e.g., host cells or isolated cells) that include a nucleic acid provided herein (e.g., a nucleic acid encoding an alpha chain and/or beta chain of a TCR provided herein, or a nucleic acid vector or viral vector provided herein). Such cells (e.g., host cells or isolated cells) that can be designed to include one or more nucleic acids provided herein can be prokaryotic cells or eukaryotic cells. Examples of prokaryotic cells that can include a nucleic acid provided herein (e.g., a nucleic acid encoding an alpha chain and/or beta chain of a TCR provided herein, or a nucleic acid vector or viral vector provided herein) include, without limitation, E. coli (e.g., Tb-1, TG-1, DH5α, XL-Blue MRF (Stratagene), SA2821, or Y1090 cells), Bacillus subtilis, Salmonella typhimurium, Serratia marcescens, or Pseudomonas (e.g., P. aeruginosa) cells.

Examples of eukaryotic cells that can include a nucleic acid provided herein (e.g., a nucleic acid encoding an alpha chain and/or beta chain of a TCR provided herein, or a nucleic acid vector or viral vector provided herein) include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, Chinese hamster ovary (CHO) cells, W138 BHK cells, COS-7 cells, and MDCK cells can be designed to include a nucleic acid provided herein (e.g., a nucleic acid encoding an alpha chain and/or beta chain of a TCR provided herein, or a nucleic acid vector or viral vector provided herein).

In some cases, a eukaryotic cell that includes a nucleic acid provided herein (e.g., a nucleic acid encoding an alpha chain and/or beta chain of a TCR provided herein, or a nucleic acid vector or viral vector provided herein) is an immune cell such as a T cell. Such cells can include an exogenous TCR, expressed from the nucleic acid provided herein, on their surface that has reactivity against the mutated form of SF3B1 without having reactivity to the non-mutated form of SF3B1. A T cell can be any type of T cell and can be of any developmental stage, including CD4⁺ and/or CD8⁺ T cells. For example, the T cells can be helper T cells, e.g., Th1 and Th2 cells, CD8⁺ T cells (e.g., cytotoxic T cells), tumor infiltrating cells (TILs), memory T cells, naive T cells, cytotoxic T lymphocytes (CTLs), regulatory T cells, activated T cells, memory T cells, or natural killer cells. The T cell can be a cultured T cell, e.g., a primary T cell or a T cell from a cultured T cell line, e.g., Jurkat or Sup-T1. In some cases, the T cell can be isolated from a mammal such as a human. Mammalian T cells can be obtained, for example, from blood, bone marrow, lymph node, the thymus, or other tissues or fluids. T cells also can be enriched or purified.

In some cases, the T cell can be isolated from the mammal to be treated, i.e., the cells are autologous to the mammal (e.g., human), and modified to include a nucleic acid provided herein (e.g., a nucleic acid encoding an alpha chain and/or beta chain of a TCR provided herein, or a nucleic acid vector or viral vector provided herein) and comprise an exogenous TCR on the surface of the cell that is expressed from the nucleic acid. For example, for autologous cells, the immune cells can be obtained from the mammal's blood, cord blood, or bone marrow. In some cases, the T cells are heterologous to the mammal, i.e., the cells are not from the mammal to be treated.

In some cases, an immune cell such as a T cell (e.g., a human T cell) that includes a nucleic acid provided herein (e.g., a nucleic acid encoding an alpha chain and/or beta chain of a TCR provided herein, or a nucleic acid vector or viral vector provided herein) and comprises an exogenous TCR expressed from the nucleic acid, can lack expression of an endogenous alpha chain of a TCR and/or lack expression of an endogenous beta chain of a TCR. Any appropriate method can be used to generate T cells that lack expression of one or both chains of an endogenous TCR. For example, gene editing techniques such as those that involve using Clustered Regularly Interspaced Short Palindromic Repeats (CRISPR) technology or Transcription Activator-Like Effector Nuclease (TALEN) technology can be used to interfere with the expression of one or both chains of an endogenous TCR.

In some cases, an immune cell such as a natural killer (NK) cell that includes a nucleic acid provided herein (e.g., a nucleic acid encoding an alpha chain and/or beta chain of a TCR provided herein, or a nucleic acid vector or viral vector provided herein) and comprises an exogenous TCR expressed from the nucleic acid, can be engineered to express one or more of the CD3 chains of the TCR complex (e.g., the CD3ε, CD3γ, CD3 ζ, and optionally CD3δ). In such cases, the exogenous TCR can be expressed on the surface of the immune cell (e.g., a NK cell) in combination with the exogenously provided one or more CD3 chains of a CD3 complex.

In some cases, an immune cell such as a T cell (e.g., a human T cell) that includes a nucleic acid provided herein (e.g., a nucleic acid encoding an alpha chain and/or beta chain of a TCR provided herein, or a nucleic acid vector or viral vector provided herein) and comprises an exogenous TCR expressed from the nucleic acid, expresses an endogenous TCR. In such cases, a portion of the TCRs present on the surface of such T cells can be endogenous TCRs, a portion of the TCRs present on the surface of such T cells can be exogenously provided TCRs (e.g., TCRs containing the alpha and beta chain polypeptides described herein), and a portion of the TCRs present on the surface of such T cells can have one endogenous provided TCR chain and one exogenously provided TCR chain.

In some cases, the constant regions of the alpha and beta chains encoded by a nucleic acid provided herein can be engineered to include sequences that encode one or more cysteine residues to increase the pairing of the alpha and beta chains with each other when expressed within a cell. Examples of such cysteine residues include, without limitation, those described elsewhere (see, e.g., Kuball et al., Blood, 109:2331-2338 (2007)).

Any appropriate method can be used to introduce one or more nucleic acids provided herein (e.g., a vector containing a nucleic acid encoding the alpha chain and/or beta chain polypeptides of a TCR provided herein) into a cell (e.g., a host cell or an isolated cell). For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a cell (e.g., a host cell or an isolated cell) (see, e.g., Green and Sambrook, Molecular Cloning: A Laboratory Manual, 4th edition, Cold Spring Harbor Laboratory, NY (2012); Davis et al., Basic Methods in Molecular Biology (1986); and Neumann et al., EMBO J., 1:841 (1982)).

The immune cells (e.g., T cells) can be activated and expanded, before or after introduction of the nucleic acid. T-cell activation reagents are commercially available, including CTS™ Dynabeads™ CD3/28, MACS GMP ExpAct Treg beads (Miltenyi Biotec), MACS GMP TransAct CD3/28 beads (Miltenyi Biotec), and Expamer technology (Juno Therapeutics). See, for example, Wang and Riviere, Molecular Therapy Oncolytics, 3:16015 (2016). T cells can be expanded, for example, using a GE WAVE bioreactor system that includes a Cellbag Bioreactor and a rocking base, and which allows the cells to expand to more than 10⁷ cells/mL. In some cases, T cells can be expanded using Gas Permeable Rapid Expansion (G-Rex®) (Wilson Wolf Manufacturing), which includes a cell culture flask with a gas-permeable membrane at the base that allows cells to grow to a high density. T cells also can be expanded using a CliniMACS Prodigy system (Miltenyi Biotec), which uses a magnetic cell separation system, and a cell cultivation device. See, for example, Wang and Riviere, Molecular Therapy Oncolytics, 3:16015 (2016).

In some cases, the immune cells (e.g., T cells) that include, for example, a vector containing a nucleic acid encoding the alpha chain and/or beta chain polypeptides of a TCR provided herein, can be cryopreserved for use at a later time. For example, the cells can be cryopreserved with dimethylsulfoxide (DMSO) and human serum albumin (HSA). In some cases, the cells can be cryopreserved with a balanced crystalloid solution (e.g., Plasma-Lyte 148), DMSO, and HSA.

In some cases, a nucleic acid provided herein, a vector provided herein, or an immune cell provided herein can be formulated as a pharmaceutical composition for administration to a mammal (e.g., a human) to treat a disorder, disease, or condition. For example, immune cells that comprise an exogenous TCR provided herein can be formulated and used for cell therapy, e.g., adoptive cell therapy. In some cases, a nucleic acid, a vector, or an immune cell provided herein can be formulated for administration to a mammal (e.g., a human to treat cancer). In some cases, a pharmaceutical composition provided herein can include a pharmaceutically acceptable carrier such as a buffer, a salt, a surfactant, a sugar, a tonicity modifier, or combinations thereof. See, for example, Gervasi, et al., Eur. J. Pharmaceutics and Biopharmaceutics, Volume 131, pages 8-24 (2018). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include immune cells comprising an exogenous TCR provided herein (or nucleic acid or vector encoding an alpha and/or beta chain of a TCR) can be formulated to include a buffer (e.g., an acetate, citrate, histidine, succinate, phosphate, hydroxymethylaminomethane (Tris), or Plasma-lyte buffer).

In some cases, when a pharmaceutical composition is formulated to include immune cells, the formulation contains a sufficient number of cells to deliver about 1×10⁵ to about 1×10¹⁰ cells/kg body weight to the mammal. For example, the formulation contains a sufficient number of cells to deliver 1×10⁵ to about 1×10⁹ cells/kg body weight, 1×10⁵ to about 1×10⁸ cells/kg body weight, 2×10⁵ to about 2×10⁶ cells/kg body weight, or 1×10⁶ to about 1×10⁷ cells/kg body weight to the mammal.

In some cases, when a pharmaceutical composition is formulated to include one or more nucleic acids (e.g., vectors such as viral vectors) encoding an alpha and/or beta chain of a TCR provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid (e.g., a vector such as a viral vector) encoding an alpha and/or beta chain of a TCR per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid (e.g., a vector such as a viral vector) encoding a an alpha and/or beta chain of a TCR.

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more nucleic acids provided herein) can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing immune cells that include an exogenous TCR provided herein (or nucleic acid or vector encoding an alpha and/or beta chain of such a TCR) to a mammal (e.g., a human). For example, a composition (e.g., a pharmaceutical composition provided herein) containing immune cells that include an exogenous TCR provided herein (or nucleic acid or vector encoding an alpha and/or beta chain of such a TCR) can be administered to a mammal (e.g., a human) in need thereof to treat a disorder, disease, or condition. Any appropriate method can be used to administer a composition (e.g., a pharmaceutical composition provided herein) to a mammal (e.g., a human). For example, a composition (e.g., a pharmaceutical composition provided herein) containing immune cells that include an exogenous TCR provided herein (or nucleic acid or vector encoding an alpha and/or beta chain of such a TCR) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) containing immune cells that include an exogenous TCR provided herein (or nucleic acid or vector encoding an alpha and/or beta chain of such a TCR) can be adjusted for the mammal being treated.

Effective doses of a composition containing immune cells that include an exogenous TCR provided herein (or nucleic acid or vector encoding an alpha and/or beta chain of such a TCR) can vary depending on the severity of the disorder, the route of administration, the age and general health condition of the subject, excipient usage, the possibility of co-usage with other therapeutic treatments such as use of other agents, and the judgment of the treating physician.

In some cases, an effective amount of a composition containing immune cells comprising an exogenous TCR provided herein (or nucleic acid or vector encoding an alpha and/or beta chain of such a TCR) can be an amount that reduces the activity of the target antigen within a mammal without producing significant toxicity to the mammal. For example, an effective amount of immune cells that comprise an exogenous TCR provided herein can be from about 1×10⁵ to about 1×10¹⁰ cells/kg body weight. For example, the cells can be administered to the mammal at a range of 1×10⁵ to about 1×10⁹ cells/kg body weight, 1×10⁵ to about 1×10⁸ cells/kg body weight, 2×10⁵ to about 2×10⁶ cells/kg body weight, 1×10⁶ to about 1×10⁷ cells/kg body weight.

The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of immune cells that include an exogenous TCR provided herein (or nucleic acid or vector an alpha and/or beta chain of such a TCR) can be any amount that maintains a fairly steady state of reduced target antigen activity within a mammal without producing significant toxicity to the mammal. For example, the frequency of administration of immune cells provided herein can be from about twice daily to about once a week. In some cases, the frequency of administration of immune cells provided herein can be daily. The frequency of administration of immune cells provided herein can remain constant or can be variable during the duration of treatment. A course of treatment with a composition containing immune cells provided herein can include rest periods. For example, a composition containing immune cells provided herein can be administered daily over a two-week period followed by a one-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing immune cells that include an exogenous TCR provided herein (or nucleic acid or vector encoding the alpha and/or beta chains of such a TCR) can be any appropriate duration that reduces the activity of the target antigen within a mammal without producing significant toxicity to the mammal. In some cases, the effective duration can vary from several weeks to several years (e.g., 5, 10, 15, or more years). Multiple factors can influence the actual effective duration used for a particular treatment. For example, an effective duration can vary with the frequency of administration, effective amount, use of multiple treatment agents, route of administration, and severity of the condition being treated.

In some cases, a composition containing immune cells that include an exogenous TCR provided herein (or nucleic acid or vector encoding the alpha and/or beta chains of such a TCR) can be administered as part of a combination treatment, such as simultaneously with or sequentially with, in any order, another therapeutic agent, such as a cytokine (e.g., IL-2), antibody (e.g., tocilizumab, which blocks IL-6 activity), an immune checkpoint inhibitor (e.g., one or more of an anti-PD-1 antibody, an anti-PDL-1 antibody, or an anti-CTLA-4 antibody), or a chemotherapeutic agent.

The invention will be further described in the following examples, which do not limit the scope of the invention described in the claims.

### EXAMPLES

To confirm the specificity and activity of nucleic acid encoding TCRs against SF3B1, the nucleic acids were introduced into donor T cells. These TCR-transduced T cells were co-cultured with dendritic cells expressing wildtype SF3B1 or mutated forms of SF3B1 (R625H, R625G, R625L, or R625C). After overnight incubation, the supernatant was assessed for production of interferon-gamma. It was found that the introduced nucleic acids encoding TCRs conferred SF3B1 R625H mutation reactivity without reactivity against the wildtype form of SF3B1 or against other mutant forms of SF3B1 (R625G, R625L, or R625C). See, FIG. 9.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-90. (canceled)

91. An isolated human immune cell comprising an exogenous T cell receptor (TCR) having affinity for a human splicing factor 3B subunit 1A (SF3B1) peptide comprising a histidine at position 625 in the context of DRB3, said exogenous TCR comprising an alpha chain and a beta chain,
said alpha chain comprising the amino acid sequences set forth in SEQ ID NO:32, SEQ ID NO:33, and SEQ ID NO:34, and
said beta chain comprising the amino acid sequences set forth in SEQ ID NO:23, SEQ ID NO:24, and SEQ ID NO:25.

92. The immune cell of claim 91, wherein said alpha chain comprises an amino sequence having at least 80 percent identity to the amino acid sequence set forth in SEQ ID NO:40 or SEQ ID NO:44.

93. The immune cell of claim 91, wherein said beta chain comprises an amino acid sequence having at least 80 percent identity to the amino acid sequence set forth in SEQ ID NO:31 or SEQ ID NO:42.

94. The immune cell of claim 91, wherein said immune cell is a T cell.

95. The immune cell of claim 94, wherein expression of the endogenous TCR alpha and beta chain coding sequences are downregulated in said T cell.

96. A nucleic acid molecule comprising (i) a nucleic acid sequence encoding an alpha chain of a TCR having affinity for a human SF3B1 peptide comprising a histidine at position 625 in the context of DRB3 and (ii) a nucleic acid sequence encoding a beta chain of said TCR,
said alpha chain comprising the amino acid sequences set forth in SEQ ID NO:32, SEQ ID NO:33, and SEQ ID NO:34, and
said beta chain comprising the amino acid sequences set forth in SEQ ID NO:23, SEQ ID NO:24, and SEQ ID NO:25.

97. The nucleic acid of claim 96, wherein said nucleic acid comprises a promoter 5′ of said nucleic acid sequence encoding said alpha chain, and comprises a promoter 5′ of said nucleic acid sequence encoding said beta chain.

98. The nucleic acid of claim 97, wherein each of said promoters is a viral 5′ long terminal repeat or a viral 3′ long terminal repeat.

99. The nucleic acid of claim 96, wherein said nucleic acid is a vector.

100. The nucleic acid of claim 99, wherein said vector is a viral vector.

101. The nucleic acid of claim 100, wherein said vector is a retroviral vector or a lentiviral vector.

102. The nucleic acid of claim 96, said nucleic acid further comprising a nucleic acid sequence encoding a linker, wherein said linker is between the nucleic acid sequence encoding the alpha chain and the nucleic acid sequence encoding the beta chain.

103. The nucleic acid of claim 102, wherein said linker is a self-cleaving peptide.

104. The nucleic acid of claim 103, wherein said self-cleaving peptide is a self-cleaving peptide of a foot-and-mouth disease virus (FMDV), an equine rhinitis A virus (ERAVO, a Thosea asigna virus (TaV), or a porcine tescho virus-1 (PTV-1).

105. The nucleic acid of claim 103, wherein said self-cleaving peptide is a P2A peptide.

106. The nucleic acid of claim 102, wherein said linker includes a furin cleavage site.

107. A method for treating a mammal in need thereof, wherein said mammal is a human having melanoma and presenting a human SF3B1 peptide comprising a histidine at position 625 in the context of DRB3, wherein said method comprises administering to the mammal an effective amount of a pharmaceutical composition comprising human immune cells comprising an exogenous T cell receptor (TCR) having affinity for said SF3B1 peptide in the context of DRB3, wherein said exogenous TCR comprises an alpha chain and a beta chain,
wherein said alpha chain comprises the amino acid sequences set forth in SEQ ID NO:32, SEQ ID NO:33, and SEQ ID NO:34, and
wherein said beta chain comprises the amino acid sequences set forth in SEQ ID NO:23, SEQ ID NO:24, and SEQ ID NO:25.

108. The method of claim 107, wherein said immune cells are autologous to said mammal.

109. The method of claim 107, wherein said immune cells are T cells.

110. The method of claim 109, wherein expression of the endogenous TCR alpha and beta chain coding sequences are downregulated in said T cells.


SEQUENCE LISTING (108 sequences)

SEQ ID NO: 1 - protein, 5 aa, synthetic construct
SNHLY

SEQ ID NO: 2 - protein, 6 aa, synthetic construct
FYNNEI

SEQ ID NO: 3 - protein, 15 aa, synthetic construct
CASSKDYRGGEKLFF

SEQ ID NO: 4 - protein, 45 aa, synthetic construct
MDTWLVCWAIFSLLKAGLTEPEVTQTPSHQVTQMGQEVILRCVPI

SEQ ID NO: 5 - protein, 17 aa, synthetic construct
FYWYRQILGQKVEFLVS

SEQ ID NO: 6 - protein, 37 aa, synthetic construct
SEKSEIFDDQFSVERPDGSNFTLKIRSTKLEDSAMYF

SEQ ID NO: 7 - protein, 9 aa, synthetic construct
GSGTQLSVL

SEQ ID NO: 8 - protein, 173 aa, synthetic construct
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 9 - protein, 307 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSKDYRGGEKLFFGSGTQLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 10 - protein, 6 aa, synthetic construct
DRGSQS

SEQ ID NO: 11 - protein, 6 aa, synthetic construct
IYSNGD

SEQ ID NO: 12 - protein, 14 aa, synthetic construct
CAALRPGGYNKLIF

SEQ ID NO: 13 - protein, 48 aa, synthetic construct
MMKSLRVLLVILWLQLSWVWSQQKEVEQNSGPLSVPEGAIASLNCTYS

SEQ ID NO: 14 - protein, 17 aa, synthetic construct
FFWYRQYSGKSPELIMS

SEQ ID NO: 15 - protein, 33 aa, synthetic construct
KEDGRFTAQLNKASQYVSLLIRDSQPSDSATYL

SEQ ID NO: 16 - protein, 10 aa, synthetic construct
GAGTRLAVHP

SEQ ID NO: 17 - protein, 137 aa, synthetic construct
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 18 - protein, 271 aa, synthetic construct
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAALRPGGYNKLIFGAGTRLAVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 19 - protein, 177 aa, synthetic construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 20 - protein, 311 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSKDYRGGEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 21 - protein, 141 aa, synthetic construct
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 275 aa, synthetic construct
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAALRPGGYNKLIFGAGTRLAVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - protein, 5 aa, synthetic construct
MNHEY

SEQ ID NO: 24 - protein, 6 aa, synthetic construct
SMNVEV

SEQ ID NO: 25 - protein, 14 aa, synthetic construct
CASRGLATNEKLFF

SEQ ID NO: 26 - protein, 45 aa, synthetic construct
MGPQLLGYVVLCLLGAGPLEAQVTQNPRYLITVTGKKLTVTCSQN

SEQ ID NO: 27 - protein, 17 aa, synthetic construct
MSWYRQDPGLGLRQIYY

SEQ ID NO: 28 - protein, 36 aa, synthetic construct
TDKGDVPEGYKVSRKEKRNFPLILESPSPNQTSLYF

SEQ ID NO: 29 - protein, 9 aa, synthetic construct
GSGTQLSVL

SEQ ID NO: 30 - protein, 173 aa, synthetic construct; identical to SEQ ID NO: 8

SEQ ID NO: 31 - protein, 305 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRGLATNEKLFFGSGTQLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 32 - protein, 6 aa, synthetic construct
DSASNY

SEQ ID NO: 33 - protein, 7 aa, synthetic construct
IRSNVGE

SEQ ID NO: 34 - protein, 12 aa, synthetic construct
CAASKGNTPLVF

SEQ ID NO: 35 - protein, 45 aa, synthetic construct
MTSIRAVFIFLWLQLDLVNGENVEQHPSTLSVQEGDSAVIKCTYS

SEQ ID NO: 36 - protein, 17 aa, synthetic construct
FPWYKQELGKGPQLIID

SEQ ID NO: 37 - protein, 33 aa, synthetic construct
KKDQRIAVTLNKTAKHFSLHITETQPEDSAVYF

SEQ ID NO: 38 - protein, 10 aa, synthetic construct
GKGTRLSVIA

SEQ ID NO: 39 - protein, 137 aa, synthetic construct; identical to SEQ ID NO: 17

SEQ ID NO: 40 - protein, 267 aa, synthetic construct
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASKGNTPLVFGKGTRLSVIANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 41 - protein, 177 aa, synthetic construct; identical to SEQ ID NO: 19

SEQ ID NO: 42 - protein, 309 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRGLATNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 43 - protein, 141 aa, synthetic construct; identical to SEQ ID NO: 21

SEQ ID NO: 44 - protein, 271 aa, synthetic construct
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASKGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 45 - protein, 134 aa, synthetic construct
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAALRPGGYNKLIFGAGTRLAVHP

SEQ ID NO: 46 - protein, 130 aa, synthetic construct
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASKGNTPLVFGKGTRLSVIA

SEQ ID NO: 47 - protein, 134 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSKDYRGGEKLFFGSGTQLSVL

SEQ ID NO: 48 - protein, 132 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRGLATNEKLFFGSGTQLSVL

SEQ ID NO: 49 - DNA, 1821 bp, synthetic construct
  source 1..1821: mol_type = other DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtaaagactaccggggaggtgaaaaactgttttttggcagtggaacccagctctctgtcttggaggacctgagaaacgtgacaccccctaaggtgagcctgttcgagccctccaaggccgagatcgccaataagcagaaggccaccctggtgtgcctggcaaggggcttctttcctgatcacgtggagctgtcttggtgggtgaacggcaaggaggtgcacagcggcgtgtgcaccgacccacaggcctataaggagagcaattattcctactgtctgtctagccggctgagagtgagcgccacattttggcacaaccctcggaatcacttcagatgccaggtgcagtttcacggcctgtctgaggaggataagtggccagagggcagcccaaagccagtgacccagaacatctccgccgaggcatggggaagagcagactgtggcatcaccagcgcctcctaccagcagggcgtgctgtccgccacaatcctgtatgagatcctgctgggcaaggccaccctgtacgccgtgctggtgagcacactggtggtcatggctatggtgaagaggaagaactccagggcaaagcggagcggaagcggagccacaaatttctctctgctgaagcaggcaggcgatgtggaggagaaccctggaccaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgccctgaggcctggtggctacaataagctgatttttggagcagggaccaggctggctgtacacccaaacatccagaatccagagcccgccgtgtatcagctgaaggacccaagatcccaggattctaccctgtgcctgttcacagactttgattctcagatcaatgtgcccaagacaatggagagcggcaccttcatcacagacaagtgcgtgctggatatgaaggctatggactccaagtctaacggcgccatcgcctggagcaatcagacctccttcacatgccaggatatctttaaggagacaaacgccacatacccttctagcgacgtgccatgtgatgccaccctgacagagaagagcttcgagacagacatgaacctgaattttcagaatctgctggtcatcgtgctgcggatcctgctgctgaaggtggctgggtttaatctgctgatgacactgcgactgtggagtagctgatga

SEQ ID NO: 50 - protein, 605 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSKDYRGGEKLFFGSGTQLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAALRPGGYNKLIFGAGTRLAVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 51 - DNA, 1821 bp, synthetic construct
  source 1..1821: mol_type = other DNA
atggacacctggctggtgtgctgggccatcttctctctgctgaaggcaggactgaccgagccagaggtgacccagacaccttcccaccaggtgacacagatgggccaggaagtgatcctgagatgcgtgccaatcagcaaccacctgtacttttattggtaccgccagatcctgggccagaaggtggagttcctggtgtccttttataacaatgagatcagcgagaagtccgagatcttcgacgatcagttttctgtggagcggcccgatggcagcaatttcaccctgaagatcagatccacaaagctggaggattctgccatgtatttttgcgcaagctccaaggactacaggggaggagagaagctgttctttggaagcggaacccagctgtccgtgctggaggacctgcgcaacgtgacaccccctaaggtgtctctgttcgagccaagcaaggccgagatcgccaataagcagaaggccaccctggtgtgcctggcaaggggcttctttcccgatcacgtggagctgtcctggtgggtgaacggcaaggaggtgcactctggcgtgtgcaccgaccctcaggcctataaggagtctaattatagctactgtctgtctagccggctgagagtgagcgccacattttggcacaacccccggaatcacttcagatgccaggtgcagtttcacggcctgtccgaggaggataagtggcctgagggctctccaaagcccgtgacccagaacatcagcgccgaggcatggggaagggcagactgtggaatcacctctgccagctaccagcagggcgtgctgtccgccacaatcctgtatgagatcctgctgggcaaggccaccctgtacgccgtgctggtgtctacactggtggtcatggctatggtgaagcggaagaacagcagggcaaagcggagcggatctggagccacaaatttctccctgctgaagcaggccggcgatgtggaggagaatcctggcccaatgatgaagagcctgagagtgctgctggtcatcctgtggctgcagctgtcctgggtgtggtctcagcagaaggaggtggagcagaacagcggaccactgtccgtgccagagggagccatcgccagcctgaattgcacctactccgacaggggcagccagtccttcttttggtatcgccagtactccggcaagtctcctgagctgatcatgagcatctattccaacggcgacaaggaggatggccggttcacagcccagctgaataaggcctctcagtacgtgagcctgctgatcagagattcccagccatctgatagcgccacctacctgtgcgccgccctgaggccaggaggctacaacaagctgatctttggagcaggaacaagactggcagtgcaccctaacatccagaatcccgagcctgccgtgtatcagctgaaggacccacggtctcaggatagcaccctgtgcctgttcacagactttgatagccagatcaatgtgcccaagacaatggagtccggcaccttcatcacagacaagtgcgtgctggatatgaaggctatggactccaagtctaacggcgccatcgcctggtccaatcagacctctttcacatgccaggatatctttaaggagaccaacgccacatacccttcctctgacgtgccatgtgatgccaccctgacagagaagagcttcgagaccgacatgaacctgaattttcagaacctgctggtcatcgtgctgaggatcctgctgctgaaggtggccggctttaatctgctgatgacactgcgcctgtggagctcctgatga

SEQ ID NO: 52 - DNA, 1845 bp, Homo sapiens
  source 1..1845: mol_type = genomic DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtaaagactaccggggaggtgaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttcagggcaaagcggagcggaagcggagccacaaatttctctctgctgaagcaggcaggcgatgtggaggagaaccctggaccaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgccctgaggcctggtggctacaataagctgatttttggagcagggaccaggctggctgtacacccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgatga

SEQ ID NO: 53 - protein, 613 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSKDYRGGEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRSGSGATNFSLLKQAGDVEENPGPMMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAALRPGGYNKLIFGAGTRLAVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - DNA, 1845 bp, synthetic construct
  source 1..1845: mol_type = other DNA
atggacacctggctggtgtgctgggccatcttttccctgctgaaggcaggactgaccgagccagaggtgacccagacaccttctcaccaggtgacacagatgggccaggaagtgatcctgagatgcgtgccaatcagcaaccacctgtacttctattggtaccgccagatcctgggccagaaggtggagttcctggtgtccttttacaacaatgagatctccgagaagtctgagatcttcgacgatcagtttagcgtggagcggcccgatggctccaactttaccctgaagatcagaagcacaaagctggaggattccgccatgtatttctgcgcaagctccaaggactacaggggaggagagaagctgttctttggctctggcacccagctgagcgtgctggaggacctgaataaggtgttcccccctgaggtggccgtgtttgagccttccgaggccgagatctctcacacccagaaggccaccctggtgtgcctggcaaccggcttctttccagatcacgtggagctgagctggtgggtgaacggcaaggaggtgcacagcggcgtgtccaccgacccacagcccctgaaggagcagcccgccctgaatgatagcagatattgcctgtctagccggctgagagtgtccgccaccttttggcagaaccctcggaatcacttcagatgtcaggtgcagttttacggcctgagcgagaatgacgagtggacccaggatagggccaagcccgtgacacagatcgtgtccgccgaggcatggggaagggcagactgcggcttcacatccgtgtcttatcagcagggcgtgctgagcgccaccatcctgtatgagatcctgctgggcaaggccacactgtacgccgtgctggtgtccgccctggtgctgatggctatggtgaagcggaaggactttagggcaaagcgcagcggatccggagcaaccaacttctctctgctgaagcaggccggcgatgtggaggagaatcctggcccaatgatgaagtccctgagagtgctgctggtcatcctgtggctgcagctgtcttgggtgtggagccagcagaaggaggtggagcagaactccggacctctgtctgtgccagagggagccatcgcctctctgaattgtacctacagcgacaggggctctcagagcttcttttggtatcgccagtactctggcaagagccctgagctgatcatgtccatctattctaacggcgacaaggaggatggcaggtttacagcccagctgaataaggccagccagtacgtgtccctgctgatccgcgacagccagccatccgattctgccacctatctgtgcgccgccctgaggccaggaggatacaacaagctgatcttcggagcaggaacaagactggcagtgcacccaaacatccagaatcccgaccctgccgtgtatcagctgagggactccaagtcctctgataagagcgtgtgcctgttcaccgactttgattctcagacaaacgtgagccagtccaaggacagcgacgtgtacatcaccgacaagacagtgctggatatgcggagcatggacttcaagtctaacagcgccgtggcctggagcaataagtccgatttcgcctgcgccaatgcctttaacaattccatcatccctgaggataccttctttccatctcccgagagctcctgtgacgtgaagctggtggagaagtctttcgagaccgatacaaacctgaattttcagaacctgagcgtgatcggcttccggatcctgctgctgaaggtggccggcttcaatctgctgatgacactgagactgtggtctagctgatga

SEQ ID NO: 55 - DNA, 1803 bp, synthetic construct
  source 1..1803: mol_type = other DNA
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagggggttagcaactaatgaaaaactgttttttggcagtggaacccagctctctgtcttggaggacctgaggaacgtgaccccacctaaggtgtccctgttcgagccttctaaggccgagatcgccaataagcagaaggccaccctggtgtgcctggcaaggggcttctttccagatcacgtggagctgagctggtgggtgaacggcaaggaggtgcactccggcgtgtgcaccgacccacaggcctacaaggagagcaattactcctattgtctgagctcccggctgagagtgtccgccacattttggcacaaccccaggaatcacttccgctgccaggtgcagtttcacggcctgagcgaggaggataagtggcctgagggctcccctaagccagtgacccagaacatctctgccgaggcatggggaagggcagactgtggaatcaccagcgcctcctatcagcagggcgtgctgagcgccacaatcctgtacgagatcctgctgggcaaggccaccctgtatgccgtgctggtgtccacactggtggtcatggctatggtgaagcggaagaactctagggcaaagcggagcggaagcggagccacaaatttctctctgctgaagcaggcaggcgatgtggaggagaaccctggaccaatgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtaaaggaaacacacctcttgtctttggaaagggcacaagactttctgtgattgcaaacatccagaatccagagcccgccgtgtatcagctgaaggacccaagatcccaggattctaccctgtgcctgttcacagactttgattctcagatcaatgtgcccaagacaatggagagcggcaccttcatcacagacaagtgcgtgctggatatgaaggctatggactccaagtctaacggcgccatcgcctggagcaatcagacctccttcacatgccaggatatctttaaggagacaaacgccacatacccttctagcgacgtgccatgtgatgccaccctgacagagaagagcttcgagacagacatgaacctgaattttcagaatctgctggtcatcgtgctgcggatcctgctgctgaaggtggctgggtttaatctgctgatgacactgcgactgtggagtagctgatga

SEQ ID NO: 56 - protein, 599 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRGLATNEKLFFGSGTQLSVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASKGNTPLVFGKGTRLSVIANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 57 - DNA, 1803 bp, synthetic construct
  source 1..1803: mol_type = other DNA
atgggaccacagctgctgggatacgtggtgctgtgcctgctgggagcaggaccactggaggcacaggtgacccagaaccctagatatctgatcaccgtgacaggcaagaagctgaccgtgacatgtagccagaacatgaatcacgagtacatgtcctggtatcggcaggatcctggcctgggcctgagacagatctactatagcatgaatgtggaggtgaccgacaagggcgatgtgcctgagggctacaaggtgtcccggaaggagaagagaaacttcccactgatcctggagagcccatcccccaatcagacctctctgtatttttgcgcaagcaggggactggcaaccaatgagaagctgttctttggctccggcacacagctgtctgtgctggaggacctgagaaacgtgaccccccctaaggtgagcctgttcgagccctccaaggccgagatcgccaataagcagaaggccaccctggtgtgcctggcaaggggcttctttcctgatcacgtggagctgtcttggtgggtgaacggcaaggaggtgcacagcggcgtgtgcaccgacccacaggcctacaaggagtctaattacagctattgtctgagctcccggctgagagtgtccgccacattttggcacaacccaaggaatcacttccgctgccaggtgcagtttcacggcctgtctgaggaggataagtggccagagggaagccctaagccagtgacccagaacatctccgccgaggcatggggaagggcagactgtggaatcacctctgccagctatcagcagggcgtgctgagcgccacaatcctgtacgagatcctgctgggcaaggccaccctgtatgccgtgctggtgagcacactggtggtcatggctatggtgaagaggaagaactccagggcaaagcggagcggatctggagccaccaatttctctctgctgaagcaggcaggcgatgtggaggagaatccaggacctatgacatccatccgcgccgtgttcatctttctgtggctgcagctggacctggtgaacggcgagaatgtggagcagcacccctccaccctgtctgtgcaggagggcgatagcgccgtgatcaagtgcacatacagcgactccgcctctaactacttcccctggtataagcaggagctgggcaagggcccccagctgatcatcgatatcaggtctaacgtgggcgagaagaaggaccagcgcatcgccgtgaccctgaataagacagccaagcactttagcctgcacatcaccgagacacagcccgaggattccgccgtgtacttctgtgccgcctctaagggcaacacccctctggtgtttggcaagggcacaaggctgtccgtgatcgccaacatccagaatccagagcccgccgtgtatcagctgaaggaccctcgcagccaggattccaccctgtgcctgttcacagactttgattcccagatcaatgtgccaaagacaatggagtctggcaccttcatcacagacaagtgcgtgctggatatgaaggctatggacagcaagtccaacggcgccatcgcctggtctaatcagaccagcttcacatgccaggatatctttaaggagaccaacgccacatacccttctagcgacgtgccatgtgatgccaccctgacagagaagagcttcgagaccgacatgaacctgaattttcagaacctgctggtcatcgtgctgcggatcctgctgctgaaggtggccggctttaatctgctgatgacactgagactgtggtcctcttgatga

SEQ ID NO: 58 - DNA, 1827 bp, Homo sapiens
  source 1..1827: mol_type = genomic DNA
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagggggttagcaactaatgaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttcagggcaaagcggagcggaagcggagccacaaatttctctctgctgaagcaggcaggcgatgtggaggagaaccctggaccaatgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtaaaggaaacacacctcttgtctttggaaagggcacaagactttctgtgattgcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgatga

SEQ ID NO: 59 - protein, 607 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRGLATNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRSGSGATNFSLLKQAGDVEENPGPMTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASKGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - DNA, 1827 bp, synthetic construct
  source 1..1827: mol_type = other DNA
atgggaccacagctgctgggatacgtggtgctgtgcctgctgggagcaggaccactggaggcacaggtgacccagaaccctcggtatctgatcaccgtgacaggcaagaagctgaccgtgacatgttcccagaacatgaatcacgagtacatgtcttggtataggcaggaccctggactgggactgagacagatctactattccatgaatgtggaggtgaccgacaagggcgatgtgcctgagggctacaaggtgtctcggaaggagaagagaaactttccactgatcctggagtctccaagccccaatcagacatccctgtatttctgcgcctctagaggcctggccaccaacgagaagctgttctttggcagcggcacacagctgtccgtgctggaggacctgaataaggtgttcccccctgaggtggccgtgtttgagccatctgaggccgagatcagccacacccagaaggccaccctggtgtgcctggcaaccggcttctttcccgatcacgtggagctgtcctggtgggtgaacggcaaggaggtgcacagcggcgtgtccacagaccctcagccactgaaggagcagcctgccctgaatgattccaggtactgcctgagctcccggctgagagtgtctgccaccttttggcagaacccaaggaatcacttccgctgtcaggtgcagttttatggcctgagcgagaacgacgagtggacccaggatcgggccaagccagtgacacagatcgtgtccgccgaggcatggggaagagcagactgcggcttcacatccgtgtcttatcagcagggcgtgctgtctgccaccatcctgtacgagatcctgctgggcaaggccacactgtatgccgtgctggtgagcgccctggtgctgatggctatggtgaagaggaaggactttagggcaaagcgcagcggatccggagcaaccaacttctctctgctgaagcaggcaggcgatgtggaggagaatccaggacctatgacaagcatccgcgccgtgttcatctttctgtggctgcagctggatctggtgaacggcgagaatgtggagcagcacccttctaccctgagcgtgcaggagggcgatagcgccgtgatcaagtgtacatactctgacagcgcctccaactactttccctggtataagcaggagctgggcaagggcccccagctgatcatcgatatcaggtccaacgtgggcgagaagaaggaccagcgcatcgccgtgaccctgaataagacagccaagcacttcagcctgcacatcaccgagacacagcccgaggactctgccgtgtacttttgcgccgccagcaagggcaatacccctctggtgttcggcaagggcacacggctgtccgtgatcgccaacatccagaatccagatcccgccgtgtatcagctgagagacagcaagtctagcgataagagcgtgtgcctgttcaccgactttgatagccagacaaacgtgtctcagagcaaggactccgacgtgtacatcaccgacaagacagtgctggatatgcggagcatggactttaagtccaactctgccgtggcctggtccaataagtctgacttcgcctgcgccaatgcctttaacaattctatcatccccgaggataccttctttcctagcccagagtcctcttgtgacgtgaagctggtggagaagagcttcgagaccgatacaaacctgaattttcagaacctgtccgtgatcggcttcaggatcctgctgctgaaggtggccggcttcaatctgctgatgaccctgcgcctgtggagctcctgatga

SEQ ID NO: 61 - protein, 25 aa, synthetic construct
MRPDIDNMDEYVHNTTARAFAVVAS

SEQ ID NO: 62 - protein, 24 aa, synthetic construct
RPDIDNMDEYVHNTTARAFAVVAS

SEQ ID NO: 63 - protein, 23 aa, synthetic construct
PDIDNMDEYVHNTTARAFAVVAS

SEQ ID NO: 64 - protein, 22 aa, synthetic construct
DIDNMDEYVHNTTARAFAVVAS

SEQ ID NO: 65 - protein, 21 aa, synthetic construct
IDNMDEYVHNTTARAFAVVAS

SEQ ID NO: 66 - protein, 20 aa, synthetic construct
DNMDEYVHNTTARAFAVVAS

SEQ ID NO: 67 - protein, 19 aa, synthetic construct
NMDEYVHNTTARAFAVVAS

SEQ ID NO: 68 - protein, 18 aa, synthetic construct
MDEYVHNTTARAFAVVAS

SEQ ID NO: 69 - protein, 17 aa, synthetic construct
DEYVHNTTARAFAVVAS

SEQ ID NO: 70 - protein, 16 aa, synthetic construct
EYVHNTTARAFAVVAS

SEQ ID NO: 71 - protein, 17 aa, synthetic construct
MRPDIDNMDEYVHNTTA

SEQ ID NO: 72 - protein, 18 aa, synthetic construct
MRPDIDNMDEYVHNTTAR

SEQ ID NO: 73 - protein, 19 aa, synthetic construct
MRPDIDNMDEYVHNTTARA

SEQ ID NO: 74 - protein, 20 aa, synthetic construct
MRPDIDNMDEYVHNTTARAF

SEQ ID NO: 75 - protein, 21 aa, synthetic construct
MRPDIDNMDEYVHNTTARAFA

SEQ ID NO: 76 - protein, 22 aa, synthetic construct
MRPDIDNMDEYVHNTTARAFAV

SEQ ID NO: 77 - protein, 23 aa, synthetic construct
MRPDIDNMDEYVHNTTARAFAVV

SEQ ID NO: 78 - protein, 24 aa, synthetic construct
MRPDIDNMDEYVHNTTARAFAVVA

SEQ ID NO: 79 - protein, 25 aa, Homo sapiens
MRPDIDNMDEYVRNTTARAFAVVAS

SEQ ID NO: 80 - protein, 22 aa, synthetic construct
GSGATNFSLLKQAGDVEENPGP

SEQ ID NO: 81 - protein, 27 aa, synthetic construct
RAKRSGSGATNFSLLKQAGDVEENPGP

SEQ ID NO: 82 - protein, 21 aa, synthetic construct
GSGEGRGSLLTCGDVEENPGP

SEQ ID NO: 83 - protein, 23 aa, synthetic construct
GSGQCTNYALLKLAGDVESNPGP

SEQ ID NO: 84 - protein, 25 aa, synthetic construct
GSGVKQTLNFDLLKLAGDVESNPGP

SEQ ID NO: 85-87 - skipped (3 entries)

SEQ ID NO: 88 - protein, 4 aa, synthetic construct
RQKR

SEQ ID NO: 89 - protein, 25 aa, synthetic construct
MRPDIDNMDEYVGNTTARAFAVVAS

SEQ ID NO: 90 - protein, 25 aa, synthetic construct
MRPDIDNMDEYVLNTTARAFAVVAS

SEQ ID NO: 91 - protein, 25 aa, synthetic construct
MRPDIDNMDEYVCNTTARAFAVVAS

SEQ ID NO: 92 - DNA, 921 bp, synthetic construct
  source 1..921: mol_type = other DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtaaagactaccggggaggtgaaaaactgttttttggcagtggaacccagctctctgtcttggaggacctgagaaacgtgacaccccctaaggtgagcctgttcgagccctccaaggccgagatcgccaataagcagaaggccaccctggtgtgcctggcaaggggcttctttcctgatcacgtggagctgtcttggtgggtgaacggcaaggaggtgcacagcggcgtgtgcaccgacccacaggcctataaggagagcaattattcctactgtctgtctagccggctgagagtgagcgccacattttggcacaaccctcggaatcacttcagatgccaggtgcagtttcacggcctgtctgaggaggataagtggccagagggcagcccaaagccagtgacccagaacatctccgccgaggcatggggaagagcagactgtggcatcaccagcgcctcctaccagcagggcgtgctgtccgccacaatcctgtatgagatcctgctgggcaaggccaccctgtacgccgtgctggtgagcacactggtggtcatggctatggtgaagaggaagaactcc

SEQ ID NO: 93 - DNA, 819 bp, synthetic construct
  source 1..819: mol_type = other DNA
atgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgccctgaggcctggtggctacaataagctgatttttggagcagggaccaggctggctgtacacccaaacatccagaatccagagcccgccgtgtatcagctgaaggacccaagatcccaggattctaccctgtgcctgttcacagactttgattctcagatcaatgtgcccaagacaatggagagcggcaccttcatcacagacaagtgcgtgctggatatgaaggctatggactccaagtctaacggcgccatcgcctggagcaatcagacctccttcacatgccaggatatctttaaggagacaaacgccacatacccttctagcgacgtgccatgtgatgccaccctgacagagaagagcttcgagacagacatgaacctgaattttcagaatctgctggtcatcgtgctgcggatcctgctgctgaaggtggctgggtttaatctgctgatgacactgcgactgtggagtagctgatga

SEQ ID NO: 94 - DNA, 921 bp, synthetic construct
  source 1..921: mol_type = other DNA
atggacacctggctggtgtgctgggccatcttctctctgctgaaggcaggactgaccgagccagaggtgacccagacaccttcccaccaggtgacacagatgggccaggaagtgatcctgagatgcgtgccaatcagcaaccacctgtacttttattggtaccgccagatcctgggccagaaggtggagttcctggtgtccttttataacaatgagatcagcgagaagtccgagatcttcgacgatcagttttctgtggagcggcccgatggcagcaatttcaccctgaagatcagatccacaaagctggaggattctgccatgtatttttgcgcaagctccaaggactacaggggaggagagaagctgttctttggaagcggaacccagctgtccgtgctggaggacctgcgcaacgtgacaccccctaaggtgtctctgttcgagccaagcaaggccgagatcgccaataagcagaaggccaccctggtgtgcctggcaaggggcttctttcccgatcacgtggagctgtcctggtgggtgaacggcaaggaggtgcactctggcgtgtgcaccgaccctcaggcctataaggagtctaattatagctactgtctgtctagccggctgagagtgagcgccacattttggcacaacccccggaatcacttcagatgccaggtgcagtttcacggcctgtccgaggaggataagtggcctgagggctctccaaagcccgtgacccagaacatcagcgccgaggcatggggaagggcagactgtggaatcacctctgccagctaccagcagggcgtgctgtccgccacaatcctgtatgagatcctgctgggcaaggccaccctgtacgccgtgctggtgtctacactggtggtcatggctatggtgaagcggaagaacagc

SEQ ID NO: 95 - DNA, 819 bp, synthetic construct
  source 1..819: mol_type = other DNA
atgatgaagagcctgagagtgctgctggtcatcctgtggctgcagctgtcctgggtgtggtctcagcagaaggaggtggagcagaacagcggaccactgtccgtgccagagggagccatcgccagcctgaattgcacctactccgacaggggcagccagtccttcttttggtatcgccagtactccggcaagtctcctgagctgatcatgagcatctattccaacggcgacaaggaggatggccggttcacagcccagctgaataaggcctctcagtacgtgagcctgctgatcagagattcccagccatctgatagcgccacctacctgtgcgccgccctgaggccaggaggctacaacaagctgatctttggagcaggaacaagactggcagtgcaccctaacatccagaatcccgagcctgccgtgtatcagctgaaggacccacggtctcaggatagcaccctgtgcctgttcacagactttgatagccagatcaatgtgcccaagacaatggagtccggcaccttcatcacagacaagtgcgtgctggatatgaaggctatggactccaagtctaacggcgccatcgcctggtccaatcagacctctttcacatgccaggatatctttaaggagaccaacgccacatacccttcctctgacgtgccatgtgatgccaccctgacagagaagagcttcgagaccgacatgaacctgaattttcagaacctgctggtcatcgtgctgaggatcctgctgctgaaggtggccggctttaatctgctgatgacactgcgcctgtggagctcctgatga

SEQ ID NO: 96 - DNA, 933 bp, Homo sapiens
  source 1..933: mol_type = genomic DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtaaagactaccggggaggtgaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 97 - DNA, 831 bp, Homo sapiens
  source 1..831: mol_type = genomic DNA
atgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgccctgaggcctggtggctacaataagctgatttttggagcagggaccaggctggctgtacacccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgatga

SEQ ID NO: 98 - DNA, 933 bp, synthetic construct
  source 1..933: mol_type = other DNA
atggacacctggctggtgtgctgggccatcttttccctgctgaaggcaggactgaccgagccagaggtgacccagacaccttctcaccaggtgacacagatgggccaggaagtgatcctgagatgcgtgccaatcagcaaccacctgtacttctattggtaccgccagatcctgggccagaaggtggagttcctggtgtccttttacaacaatgagatctccgagaagtctgagatcttcgacgatcagtttagcgtggagcggcccgatggctccaactttaccctgaagatcagaagcacaaagctggaggattccgccatgtatttctgcgcaagctccaaggactacaggggaggagagaagctgttctttggctctggcacccagctgagcgtgctggaggacctgaataaggtgttcccccctgaggtggccgtgtttgagccttccgaggccgagatctctcacacccagaaggccaccctggtgtgcctggcaaccggcttctttccagatcacgtggagctgagctggtgggtgaacggcaaggaggtgcacagcggcgtgtccaccgacccacagcccctgaaggagcagcccgccctgaatgatagcagatattgcctgtctagccggctgagagtgtccgccaccttttggcagaaccctcggaatcacttcagatgtcaggtgcagttttacggcctgagcgagaatgacgagtggacccaggatagggccaagcccgtgacacagatcgtgtccgccgaggcatggggaagggcagactgcggcttcacatccgtgtcttatcagcagggcgtgctgagcgccaccatcctgtatgagatcctgctgggcaaggccacactgtacgccgtgctggtgtccgccctggtgctgatggctatggtgaagcggaaggacttt

SEQ ID NO: 99 - DNA, 831 bp, synthetic construct
  source 1..831: mol_type = other DNA
atgatgaagtccctgagagtgctgctggtcatcctgtggctgcagctgtcttgggtgtggagccagcagaaggaggtggagcagaactccggacctctgtctgtgccagagggagccatcgcctctctgaattgtacctacagcgacaggggctctcagagcttcttttggtatcgccagtactctggcaagagccctgagctgatcatgtccatctattctaacggcgacaaggaggatggcaggtttacagcccagctgaataaggccagccagtacgtgtccctgctgatccgcgacagccagccatccgattctgccacctatctgtgcgccgccctgaggccaggaggatacaacaagctgatcttcggagcaggaacaagactggcagtgcacccaaacatccagaatcccgaccctgccgtgtatcagctgagggactccaagtcctctgataagagcgtgtgcctgttcaccgactttgattctcagacaaacgtgagccagtccaaggacagcgacgtgtacatcaccgacaagacagtgctggatatgcggagcatggacttcaagtctaacagcgccgtggcctggagcaataagtccgatttcgcctgcgccaatgcctttaacaattccatcatccctgaggataccttctttccatctcccgagagctcctgtgacgtgaagctggtggagaagtctttcgagaccgatacaaacctgaattttcagaacctgagcgtgatcggcttccggatcctgctgctgaaggtggccggcttcaatctgctgatgacactgagactgtggtctagctgatga

SEQ ID NO: 100 - DNA, 915 bp, synthetic construct
  source 1..915: mol_type = other DNA
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagggggttagcaactaatgaaaaactgttttttggcagtggaacccagctctctgtcttggaggacctgaggaacgtgaccccacctaaggtgtccctgttcgagccttctaaggccgagatcgccaataagcagaaggccaccctggtgtgcctggcaaggggcttctttccagatcacgtggagctgagctggtgggtgaacggcaaggaggtgcactccggcgtgtgcaccgacccacaggcctacaaggagagcaattactcctattgtctgagctcccggctgagagtgtccgccacattttggcacaaccccaggaatcacttccgctgccaggtgcagtttcacggcctgagcgaggaggataagtggcctgagggctcccctaagccagtgacccagaacatctctgccgaggcatggggaagggcagactgtggaatcaccagcgcctcctatcagcagggcgtgctgagcgccacaatcctgtacgagatcctgctgggcaaggccaccctgtatgccgtgctggtgtccacactggtggtcatggctatggtgaagcggaagaactct

SEQ ID NO: 101 - DNA, 807 bp, synthetic construct
  source 1..807: mol_type = other DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtaaaggaaacacacctcttgtctttggaaagggcacaagactttctgtgattgcaaacatccagaatccagagcccgccgtgtatcagctgaaggacccaagatcccaggattctaccctgtgcctgttcacagactttgattctcagatcaatgtgcccaagacaatggagagcggcaccttcatcacagacaagtgcgtgctggatatgaaggctatggactccaagtctaacggcgccatcgcctggagcaatcagacctccttcacatgccaggatatctttaaggagacaaacgccacatacccttctagcgacgtgccatgtgatgccaccctgacagagaagagcttcgagacagacatgaacctgaattttcagaatctgctggtcatcgtgctgcggatcctgctgctgaaggtggctgggtttaatctgctgatgacactgcgactgtggagtagctgatga

SEQ ID NO: 102 - DNA, 915 bp, synthetic construct
  source 1..915: mol_type = other DNA
atgggaccacagctgctgggatacgtggtgctgtgcctgctgggagcaggaccactggaggcacaggtgacccagaaccctagatatctgatcaccgtgacaggcaagaagctgaccgtgacatgtagccagaacatgaatcacgagtacatgtcctggtatcggcaggatcctggcctgggcctgagacagatctactatagcatgaatgtggaggtgaccgacaagggcgatgtgcctgagggctacaaggtgtcccggaaggagaagagaaacttcccactgatcctggagagcccatcccccaatcagacctctctgtatttttgcgcaagcaggggactggcaaccaatgagaagctgttctttggctccggcacacagctgtctgtgctggaggacctgagaaacgtgaccccccctaaggtgagcctgttcgagccctccaaggccgagatcgccaataagcagaaggccaccctggtgtgcctggcaaggggcttctttcctgatcacgtggagctgtcttggtgggtgaacggcaaggaggtgcacagcggcgtgtgcaccgacccacaggcctacaaggagtctaattacagctattgtctgagctcccggctgagagtgtccgccacattttggcacaacccaaggaatcacttccgctgccaggtgcagtttcacggcctgtctgaggaggataagtggccagagggaagccctaagccagtgacccagaacatctccgccgaggcatggggaagggcagactgtggaatcacctctgccagctatcagcagggcgtgctgagcgccacaatcctgtacgagatcctgctgggcaaggccaccctgtatgccgtgctggtgagcacactggtggtcatggctatggtgaagaggaagaactcc

SEQ ID NO: 103 - DNA, 807 bp, synthetic construct
  source 1..807: mol_type = other DNA
atgacatccatccgcgccgtgttcatctttctgtggctgcagctggacctggtgaacggcgagaatgtggagcagcacccctccaccctgtctgtgcaggagggcgatagcgccgtgatcaagtgcacatacagcgactccgcctctaactacttcccctggtataagcaggagctgggcaagggcccccagctgatcatcgatatcaggtctaacgtgggcgagaagaaggaccagcgcatcgccgtgaccctgaataagacagccaagcactttagcctgcacatcaccgagacacagcccgaggattccgccgtgtacttctgtgccgcctctaagggcaacacccctctggtgtttggcaagggcacaaggctgtccgtgatcgccaacatccagaatccagagcccgccgtgtatcagctgaaggaccctcgcagccaggattccaccctgtgcctgttcacagactttgattcccagatcaatgtgccaaagacaatggagtctggcaccttcatcacagacaagtgcgtgctggatatgaaggctatggacagcaagtccaacggcgccatcgcctggtctaatcagaccagcttcacatgccaggatatctttaaggagaccaacgccacatacccttctagcgacgtgccatgtgatgccaccctgacagagaagagcttcgagaccgacatgaacctgaattttcagaacctgctggtcatcgtgctgcggatcctgctgctgaaggtggccggctttaatctgctgatgacactgagactgtggtcctcttgatga

SEQ ID NO: 104 - DNA, 927 bp, Homo sapiens
  source 1..927: mol_type = genomic DNA
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagggggttagcaactaatgaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 105 - DNA, 819 bp, Homo sapiens
  source 1..819: mol_type = genomic DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtaaaggaaacacacctcttgtctttggaaagggcacaagactttctgtgattgcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgatga

SEQ ID NO: 106 - DNA, 927 bp, synthetic construct
  source 1..927: mol_type = other DNA
atgggaccacagctgctgggatacgtggtgctgtgcctgctgggagcaggaccactggaggcacaggtgacccagaaccctcggtatctgatcaccgtgacaggcaagaagctgaccgtgacatgttcccagaacatgaatcacgagtacatgtcttggtataggcaggaccctggactgggactgagacagatctactattccatgaatgtggaggtgaccgacaagggcgatgtgcctgagggctacaaggtgtctcggaaggagaagagaaactttccactgatcctggagtctccaagccccaatcagacatccctgtatttctgcgcctctagaggcctggccaccaacgagaagctgttctttggcagcggcacacagctgtccgtgctggaggacctgaataaggtgttcccccctgaggtggccgtgtttgagccatctgaggccgagatcagccacacccagaaggccaccctggtgtgcctggcaaccggcttctttcccgatcacgtggagctgtcctggtgggtgaacggcaaggaggtgcacagcggcgtgtccacagaccctcagccactgaaggagcagcctgccctgaatgattccaggtactgcctgagctcccggctgagagtgtctgccaccttttggcagaacccaaggaatcacttccgctgtcaggtgcagttttatggcctgagcgagaacgacgagtggacccaggatcgggccaagccagtgacacagatcgtgtccgccgaggcatggggaagagcagactgcggcttcacatccgtgtcttatcagcagggcgtgctgtctgccaccatcctgtacgagatcctgctgggcaaggccacactgtatgccgtgctggtgagcgccctggtgctgatggctatggtgaagaggaaggacttt

SEQ ID NO: 107 - DNA, 819 bp, synthetic construct
  source 1..819: mol_type = other DNA
atgacaagcatccgcgccgtgttcatctttctgtggctgcagctggatctggtgaacggcgagaatgtggagcagcacccttctaccctgagcgtgcaggagggcgatagcgccgtgatcaagtgtacatactctgacagcgcctccaactactttccctggtataagcaggagctgggcaagggcccccagctgatcatcgatatcaggtccaacgtgggcgagaagaaggaccagcgcatcgccgtgaccctgaataagacagccaagcacttcagcctgcacatcaccgagacacagcccgaggactctgccgtgtacttttgcgccgccagcaagggcaatacccctctggtgttcggcaagggcacacggctgtccgtgatcgccaacatccagaatccagatcccgccgtgtatcagctgagagacagcaagtctagcgataagagcgtgtgcctgttcaccgactttgatagccagacaaacgtgtctcagagcaaggactccgacgtgtacatcaccgacaagacagtgctggatatgcggagcatggactttaagtccaactctgccgtggcctggtccaataagtctgacttcgcctgcgccaatgcctttaacaattctatcatccccgaggataccttctttcctagcccagagtcctcttgtgacgtgaagctggtggagaagagcttcgagaccgatacaaacctgaattttcagaacctgtccgtgatcggcttcaggatcctgctgctgaaggtggccggcttcaatctgctgatgaccctgcgcctgtggagctcctgatga

SEQ ID NO: 108 - skipped